(19) United States
(12) Patent Application Publication
(10) Pub. No.: US 2019/0038151 A1
(43) Pub. Date: Feb. 7, 2019

(54) ELECTRONIC DEVICE FOR DETERMINING BIOMETRIC INFORMATION AND METHOD OF OPERATING SAME

(71) Applicant: Samsung Electronics Co., Ltd., Suwon-si (KR)

(72) Inventors: Dong-Hyun LEE, Yongin-si (KR); Ik-Joo BYUN, Suwon-si (KR); Seung-Hwan SHIN, Suwon-si (KR); Jun-Seok OH, Suwon-si (KR); Dong-Wook KIM, Seoul (KR); Jong-Min CHOI, Seoul (KR); Tae-Ho KIM, Cheongju-si (KR); Seung-Eun LEE, Seoul (KR)

(21) Appl. No.: 16/044,921

(22) Filed: Jul. 25, 2018

(30) Foreign Application Priority Data
Aug. 1, 2017 (KR) 10-2017-0097767

(51) Int. Cl. A61B 5/021; A61B 5/024; G06T 7/00; G06T 7/11; A61B 5/00

(52) U.S. Cl. CPC A61B 5/02108; A61B 5/02416; G06T 2207/30101; G06T 7/11; A61B 5/7235; G06T 7/0016

(57) ABSTRACT

An electronic device includes a first sensor, a camera, and a processor functionally connected to the first sensor and the camera, wherein the processor is configured to acquire a first biometric signal through the first sensor and a second biometric signal through the camera at a first location, acquire a third biometric signal through the first sensor and a fourth biometric signal through the camera at a second location, and determine a blood pressure based on the first biometric signal and the second biometric signal acquired at the first location and the third biometric signal and the fourth biometric signal acquired at the second location.

### CROSS-REFERENCE TO RELATED APPLICATION AND CLAIM OF PRIORITY

This application claims the priority under 35 U.S.C. § 119(a) to Korean Patent Application Serial No. 10-2017-0097767, which was filed in the Korean Intellectual Property Office on Aug. 1, 2017, the entire content of which is hereby incorporated by reference.

### BACKGROUND

### 1. Field

The present disclosure relates to an electronic device for determining biometric information and a method of operating the same.

### 2. Description of Related Art

Recently, electronic devices including a sensor that can measure user's biometric information have developed. A user may measure information related to a user's body through an electronic device to thus learn about his/her body state.

The electronic device may measure various pieces of biometric information such as a heart rate, oxygen saturation, stress, and blood pressure of the user through the sensor. For example, the electronic device may sense the part of the user's body through the sensor. The electronic device may measure various pieces of user's biometric information based on sensing information acquired through the sensor.

In order to measure blood pressure through the electronic device, a separate device (for example, an additional sensor) is required. Further, in order to measure the blood pressure through the separate device, an electrode included in the separate device must be brought into contact with the part of the user's body, which is inconvenient.

### SUMMARY

According to various embodiments, an electronic device for determining an accurate blood pressure value through a sensor and a camera included in the electronic device, and a method of operating the same, may be provided.

In accordance with an aspect of the present disclosure, an electronic device is provided. The electronic device includes a first sensor, a camera, and a processor functionally connected to the first sensor and the camera, wherein the processor is configured to acquire a first biometric signal through the first sensor and a second biometric signal through the camera at a first location, acquire a third biometric signal through the first sensor and a fourth biometric signal through the camera at a second location, and determine a blood pressure based on the first biometric signal and the second biometric signal acquired at the first location and the third biometric signal and the fourth biometric signal acquired at the second location.

In accordance with another aspect of the present disclosure, a method of operating an electronic device is provided. The method includes acquiring a first biometric signal through the first sensor included in the electronic device and a second biometric signal through the camera included in the electronic device at a first location; acquiring a third biometric signal through the first sensor and a fourth biometric signal through the camera at a second location; and determining a blood pressure based on the first biometric signal and the second biometric signal acquired at the first location and the third biometric signal and the fourth biometric signal acquired at the second location.

An electronic device according to various embodiments has an effect of acquiring Pulse Transit Times (PTTs) through photoplethysmography PPG signals acquired using a camera and a sensor at different heights and determining more accurate blood pressure based on the PTTs acquired at the different heights.

Before undertaking the DETAILED DESCRIPTION below, it may be advantageous to set forth definitions of certain words and phrases used throughout this patent document: the terms “include” and “comprise,” as well as derivatives thereof, mean inclusion without limitation; the term “or,” is inclusive, meaning and/or; the phrases “associated with” and “associated therewith,” as well as derivatives thereof, may mean to include, be included within, interconnect with, contain, be contained within, connect to or with, couple to or with, be communicable with, cooperate with, interleave, juxtapose, be proximate to, be bound to or with, have, have a property of, or the like; and the term “controller” means any device, system or part thereof that controls at least one operation, such a device may be implemented in hardware, firmware or software, or some combination of at least two of the same. It should be noted that the functionality associated with any particular controller may be centralized or distributed, whether locally or remotely.

Moreover, various functions described below can be implemented or supported by one or more computer programs, each of which is formed from computer readable program code and embodied in a computer readable medium. The terms “application” and “program” refer to one or more computer programs, software components, sets of instructions, procedures, functions, objects, classes, instances, related data, or a portion thereof adapted for implementation in a suitable computer readable program code. The phrase “computer readable program code” includes any type of computer code, including source code, object code, and executable code. The phrase “computer readable medium” includes any type of medium capable of being accessed by a computer, such as read only memory (ROM), random access memory (RAM), a hard disk drive, a compact disc (CD), a digital video disc (DVD), or any other type of memory. A “non-transitory” computer readable medium excludes wired, wireless, optical, or other communication links that transport transitory electrical or other signals. A non-transitory computer readable medium includes media where data can be permanently stored and media where data can be stored and later overwritten, such as a rewritable optical disc or an erasable memory device.

Definitions for certain words and phrases are provided throughout this patent document, those of ordinary skill in the art should understand that in many, if not most instances, such definitions apply to prior, as well as future uses of such defined words and phrases.

### BRIEF DESCRIPTION OF THE DRAWINGS

The above and other aspects, features, and advantages of the present disclosure will be more apparent from the following detailed description taken in conjunction with the accompanying drawings, in which:

FIG. 1 illustrates, in block diagram format, an electronic device and a network according to various embodiments;

FIG. 2 illustrates, in block diagram format, an electronic device according to various embodiments;

FIG. 3 illustrates, in block diagram format, a program module according to various embodiments;

FIG. 4A illustrates, in block diagram format, an electronic device according to various embodiments;

FIG. 4B illustrates operation of a processor according to embodiments of this disclosure, for example the processor illustrated in FIG. 4A;

FIGS. 5A and 5B illustrate operations of an electronic device according to various embodiments;

FIG. 6 is a flowchart illustrate operations of an electronic device according to various embodiments;

FIG. 7 illustrates operations of an electronic device according to various embodiments;

FIG. 8 illustrates aspects of acquiring first biometric information based on a first biometric signal and a second biometric signal according to various embodiments;

FIG. 9 illustrates aspects of acquiring biometric information at a first location and a second location according to various embodiments;

FIG. 10 illustrates operations of a method for determining a height difference between the first location and the second location according to various embodiments;

FIG. 11 illustrates operations of a method for determining a blood pressure based on first biometric information and second biometric information according to various embodiments;

FIGS. 12A to 12D illustrate aspects of determining the blood pressure based on first biometric information and second biometric information according to various embodiments;

FIG. 13 illustrate operations of a method of acquiring biometric signals at the first location and the second location according to various embodiments;

FIGS. 14A to 14E illustrate user interfaces for describing the operation of measuring the blood pressure according to various embodiments; and

FIGS. 15A to 15C illustrate user interfaces for describing the operation of storing the blood pressure according to various embodiments.

### DETAILED DESCRIPTION

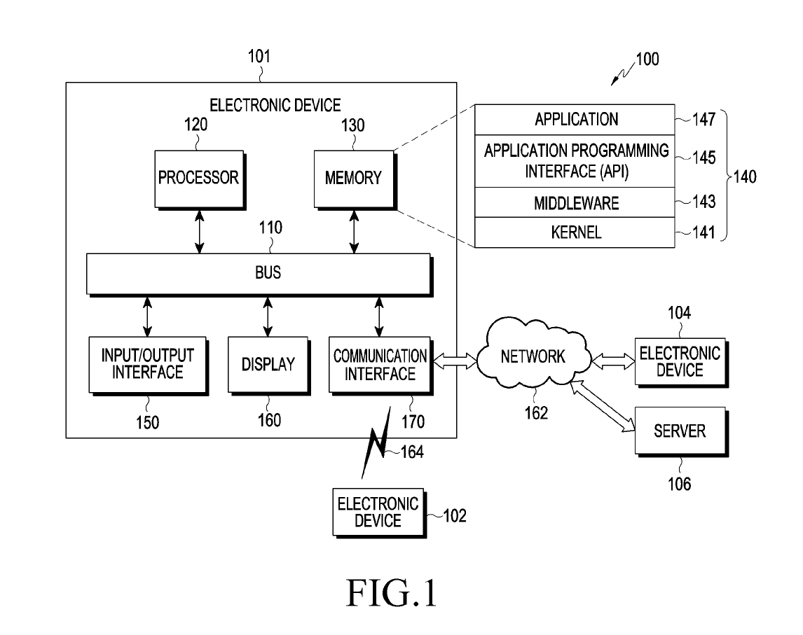

FIGS. 1 through 15C, discussed below, and the various embodiments used to describe the principles of the present disclosure in this patent document are by way of illustration only and should not be construed in any way to limit the scope of the disclosure. Those skilled in the art will understand that the principles of the present disclosure may be implemented in any suitably arranged system or device.

Hereinafter, various embodiments will be described with reference to the accompanying drawings. The embodiments and the terms used therein are not intended to limit the technology disclosed herein to specific forms, and should be understood to include various modifications, equivalents, and/or alternatives to the corresponding embodiments. In describing the drawings, similar reference numerals may be used to designate similar constituent elements. A singular expression may include a plural expression unless they are definitely different in a context. As used herein, singular forms may include plural forms as well unless the context clearly indicates otherwise. The expression “a first”, “a second”, “the first”, or “the second” used in various embodiments may modify various components regardless of the order and/or the importance but does not limit the corresponding components. When an element (e.g., first element) is referred to as being “(functionally or communicatively) connected,” or “directly coupled” to another element (second element), the element may be connected directly to the another element or connected to the another element through yet another element (e.g., third element).

The expression “configured to” as used in various embodiments may be interchangeably used with, for example, “suitable for”, “having the capacity to”, “designed to”, “adapted to”, “made to”, or “capable of” in terms of hardware or software, according to circumstances. Alternatively, in some situations, the expression “device configured to” may mean that the device, together with other devices or components, “is able to”. For example, the phrase “processor adapted (or configured) to perform A, B, and C” may mean a dedicated processor (e.g., embedded processor) only for performing the corresponding operations or a generic-purpose processor (e.g., Central Processing Unit (CPU) or Application Processor (AP)) that can perform the corresponding operations by executing one or more software programs stored in a memory device.

An electronic device according to various embodiments may include at least one of, for example, a smart phone, a tablet Personal Computer (PC), a mobile phone, a video phone, an electronic book reader (e-book reader), a desktop PC, a laptop PC, a netbook computer, a workstation, a server, a Personal Digital Assistant (PDA), a Portable Multimedia Player (PMP), a MPEG-1 audio layer-3 (MP3) player, a mobile medical device, a camera, and a wearable device. According to various embodiments, the wearable device may include at least one of an accessory type (e.g., a watch, a ring, a bracelet, an anklet, a necklace, a glasses, a contact lens, or a Head-Mounted Device (HMD)), a fabric or clothing integrated type (e.g., an electronic clothing), a body-mounted type (e.g., a skin pad, or tattoo), and a bio-implantable type (e.g., an implantable circuit). In some embodiments, the electronic device may include at least one of, for example, a television, a Digital Video Disk (DVD) player, an audio, a refrigerator, an air conditioner, a vacuum cleaner, an oven, a microwave oven, a washing machine, an air cleaner, a set-top box, a home automation control panel, a security control panel, a TV box (e.g., Samsung HOMESYNC™, APPLE TV™, OR GOOGLE TV™), a game console (e.g., XBOX™ and PLAYSTATION™), an electronic dictionary, an electronic key, a camcorder, and an electronic photo frame.

In other embodiments, the electronic device may include at least one of various medical devices (e.g., various portable medical measuring devices (a blood glucose monitoring device, a heart rate monitoring device, a blood pressure measuring device, a body temperature measuring device, etc.), a Magnetic Resonance Angiography (MRA), a Magnetic Resonance Imaging (MRI), a Computed Tomography (CT) machine, and an ultrasonic machine), a navigation device, a Global Positioning System (GPS) receiver, an Event Data Recorder (EDR), a Flight Data Recorder (FDR), a Vehicle Infotainment Devices, an electronic devices for a ship (e.g., a navigation device for a ship, and a gyro-compass), avionics, security devices, an automotive head unit, a robot for home or industry, an Automatic Teller's Machine (ATM) in banks, Point Of Sales (POS) in a shop, or internet device of things (e.g., a light bulb, various sensors, electric or gas meter, a sprinkler device, a fire alarm, a thermostat, a streetlamp, a toaster, a sporting goods, a hot water tank, a heater, a boiler, etc.). According to some embodiments, the electronic device may include at least one of a part of furniture or a building/structure, an electronic board, an electronic signature receiving device, a projector, and various kinds of measuring instruments (e.g., a water meter, an electric meter, a gas meter, and a radio wave meter). In various embodiments, the electronic device may be flexible, or may be a combination of one or more of the aforementioned various devices. The electronic device according to one embodiment is not limited to the above described devices. In the present disclosure, the term “user” may indicate a person using an electronic device or a device (e.g., an artificial intelligence electronic device) using an electronic device.

An electronic device 101 in a network environment 100 according to various embodiments will be described with reference to the non-limiting example of FIG. 1. The electronic device 101 may include a bus 110, a processor 120, a memory 130, an input/output interface 150, a display 160, and a communication interface 170. In some embodiments, the electronic device 101 may omit at least one of the above elements, or may further include other elements. The bus 110 may include a circuit that interconnects the elements 110 to 170 and transmits communication (for example, control messages or data) between the elements. The processor 120 may include one or more of a central processing unit, an application processor, and a communication processor (CP). The processor 120, for example, may carry out operations or data processing relating to the control and/or communication of at least one other element of the electronic device 101.

The memory 130 may include a volatile and/or nonvolatile memory. The memory 130 may store, for example, instructions or data relevant to at least one other element of the electronic device 101. According to an embodiment, the memory 130 may store software and/or a program 140. The program 140 may include, for example, a kernel 141, middleware 143, an Application Programming Interface (API) 145, and/or applications (or “apps”) 147. At least some of the kernel 141, the middleware 143, and the API 145 may be referred to as an operating system. The kernel 141 may control or manage system resources (for example, the bus 110, the processor 120, or the memory 130) used for executing an operation or function implemented by other programs (for example, the middleware 143, the API 145, or the application 147). Furthermore, the kernel 141 may provide an interface through which the middleware 143, the API 145, or the applications 147 may access the individual elements of the electronic device 101 to control or manage system resources.

The middleware 143 may function as, for example, an intermediary for allowing the API 145 or the applications 147 to communicate with the kernel 141 to exchange data. Furthermore, the middleware 143 may process one or more task requests, which are received from the applications 147, according to priorities thereof. For example, the middleware 143 may assign priorities for using system resources (for example, the bus 110, the processor 120, the memory 130, or the like) of the electronic device 101 to one or more of the applications 147, and may process the one or more task requests. The API 145 is an interface through which the applications 147 control functions provided from the kernel 141 or the middleware 143, and may include, for example, at least one interface or function (for example, instruction) for file control, window control, image processing, or text control. The input/output interface 150 may forward, for example, instructions or data, input from a user or an external device, to the other element(s) of the electronic device 101, or may output instructions or data, received from the other element(s) of the electronic device 101, to the user or the external device.

The display 160 may include, for example, a Liquid Crystal Display (LCD), a Light-Emitting Diode (LED) display, an Organic Light-Emitting Diode (OLED) display, a Micro Electro Mechanical System (MEMS) display, or an electronic paper display. The display 160 may display, for example, various types of content (for example, text, images, videos, icons, and/or symbols) for a user. The display 160 may include a touch screen and may receive, for example, a touch, gesture, proximity, or hovering input using an electronic pen or the user's body part. The communication interface 170 may set, for example, communication between the electronic device 101 and an external device (for example, a first external electronic device 102, a second external electronic device 104, or a server 106). For example, the communication interface 170 may be connected to a network 162 through wireless or wired communication to communicate with the external device (for example, the second external electronic device 104 or the server 106).

The wireless communication may include, for example, cellular communication that uses at least one of LTE, LTE-Advanced (LTE-A), code division multiple access (CDMA), wideband CDMA (WCDMA), universal mobile telecommunications system (UMTS), wireless broadband (WiBro), global system for mobile communications (GSM), or the like. According to an embodiment, like the short-range communication 164 illustrated in FIG. 1, the wireless communication may include, for example, at least one of Wi-Fi, Li-Fi (Light Fidelity), Bluetooth, Bluetooth Low Energy (BLE), ZigBee, Near Field Communication (NFC), magnetic secure transmission, Radio Frequency (RF), and Body Area Network (BAN). According to an embodiment, the wireless communication may include a GNSS. The GNSS may be, for example, a Global Positioning System (GPS), a Global navigation satellite system (GLONASS), a BeiDou navigation satellite system (hereinafter, referred to as “BeiDou”), or Galileo (the European global satellite-based navigation system). Hereinafter, in this document, the term “GPS” may be interchangeable with the term “GNSS”. The wired communication may include, for example, at least one of a Universal Serial Bus (USB), a High-Definition Multimedia Interface (HDMI), Recommended Standard 232 (RS-232), a Plain Old Telephone Service (POTS), and the like. The network 162 may include a telecommunications network, for example, at least one of a computer network (for example, a LAN or a WAN), the Internet, and a telephone network.

Each of the first and second external electronic devices 102 and 104 may be of a type identical to or different from that of the electronic device 101. According to various embodiments, all or some of the operations executed by the electronic device 101 may be executed by another electronic device, a plurality of electronic devices (for example, the electronic devices 102 and 104), or the server 106. According to an embodiment, when the electronic device 101 has to perform a function or service automatically or in response to a request, the electronic device 101 may request another device (for example, the electronic device 102 or 104, or the server 106) to perform at least some functions relating thereto, instead of autonomously or additionally performing the function or service. Another electronic device (for example, the electronic device 102 or 104, or the server 106) may execute the requested functions or the additional functions, and may deliver the result of execution thereof to the electronic device 101. The electronic device 101 may provide the received result as it is, or may additionally process the received result in order to provide the requested functions or services. To this end, for example, cloud-computing, distributed-computing, or client-server-computing technology may be used.

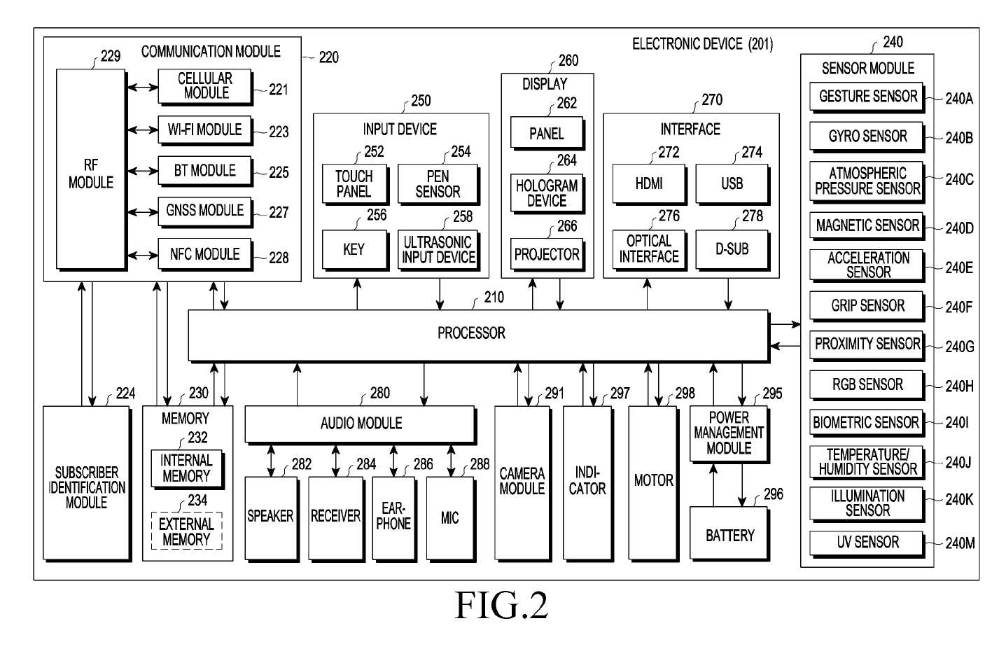

FIG. 2 illustrates an electronic device 201 according to various embodiments. The electronic device 201 may include, for example, all or part of the electronic device 101 illustrated in FIG. 1. The electronic device 201 may include at least one processor 210 (for example, an AP), a communication module 220, a subscriber identification module 224, a memory 230, a sensor module 240, an input device 250, a display 260, an interface 270, an audio module 280, a camera module 291, a power management module 295, a battery 296, an indicator 297, and a motor 298. The processor 210 may control a plurality of hardware or software elements connected to the processor 210 by running, for example, an Operating System (OS) or an application, and may perform processing and arithmetic operations of various types of data. The processor 210 may be implemented by, for example, a System on Chip (SoC). According to an embodiment, the processor 210 may further include a graphic processing unit (GPU) and/or an image signal processor. The processor 210 may also include at least some of the elements illustrated in FIG. 2 (for example, a cellular module 221). The processor 210 may load, in volatile memory, instructions or data received from at least one of the other elements (for example, nonvolatile memory), process the loaded instructions or data, and store the resultant data in the nonvolatile memory.

The communication module 220 may have a configuration identical or similar to that of the communication interface 170 illustrated in FIG. 1. The communication module 220 may include, for example, a cellular module 221, a Wi-Fi module 223, a Bluetooth module 225, a GNSS module 227, an NFC module 228, and an RF module 229. The cellular module 221 may provide, for example, a voice call, a video call, a text message service, an Internet service, or the like through a communication network. According to an embodiment, the cellular module 221 may identify or authenticate an electronic device 201 in the communication network using a subscriber identification module (for example, a Subscriber Identity Module (SIM) card) 224. According to an embodiment, the cellular module 221 may perform at least some of the functions that the AP 210 may provide. According to an embodiment, the cellular module 221 may include a communication processor (CP). In some embodiments, at least some (two or more) of the cellular module 221, the Wi-Fi module 223, the Bluetooth module 225, the GNSS module 227, and the NFC module 228 may be included in a single Integrated Chip (IC) or IC package. The RF module 229 may transmit/receive, for example, a communication signal (for example, an RF signal). The RF module 229 may include, for example, a transceiver, a Power Amp Module (PAM), a frequency filter, a Low-Noise Amplifier (LNA), an antenna, or the like. According to another embodiment, at least one of the cellular module 221, the Wi-Fi module 223, the BT module 225, the GPS module 227, and the NFC module 228 may transmit/receive an RF signal through a separate RF module. The subscriber identification module 224 may include, for example, a card that includes a subscriber identity module and/or an embedded SIM, and may contain unique identification information (for example, an Integrated Circuit Card Identifier (ICCID)) or subscriber information (for example, an International Mobile Subscriber Identity (IMSI)).

The memory 230 (for example, the memory 130) may include, for example, an internal memory 232 or an external memory 234. The internal memory 232 may include, for example, at least one of a volatile memory (for example, a DRAM, an SRAM, an SDRAM, or the like) and a nonvolatile memory (for example, a One-Time Programmable ROM (OTPROM), a PROM, an EPROM, an EEPROM, a mask ROM, a flash ROM, a flash memory, a hard disc drive, or a Solid-State Drive (SSD)). The external memory 234 may include a flash drive, for example, a compact flash (CF), a secure digital (SD), a Micro-SD, a Mini-SD, an eXtreme digital (xD), a multimedia card (MMC), a memory stick, and the like. The external memory 234 may be functionally and/or physically connected to the electronic device 201 through any of various interfaces.

The sensor module 240 may measure, for example, a physical quantity or detect the operating state of the electronic device 201, and may convert the measured or detected information into an electrical signal. The sensor module 240 may include, for example, at least one of a gesture sensor 240A, a gyro sensor 240B, an atmospheric pressure sensor 240C, a magnetic sensor 240D, an acceleration sensor 240E, a grip sensor 240F, a proximity sensor 240G, a color sensor 240H (for example, a red, green, blue (RGB) sensor), a biometric sensor 240I, a temperature/humidity sensor 240J, an illumination sensor 240K, and an ultraviolet (UV) sensor 240M. Additionally or alternatively, the sensor module 240 may include, for example, an e-nose sensor, an electromyography (EMG) sensor, an electroencephalogram (EEG) sensor, an electrocardiogram (ECG) sensor, an infrared (IR) sensor, an iris sensor, and/or a fingerprint sensor. The sensor module 240 may further include a control circuit for controlling one or more sensors included therein. In some embodiments, the electronic device 201 may further include a processor, which is configured to control the sensor module 240, as a part of the processor 210 or separately from the processor 210 in order to control the sensor module 240 while the processor 210 is in a sleep state.

The input device 250 may include, for example, a touch panel 252, a (digital) pen sensor 254, a key 256, or an ultrasonic input device 258. The touch panel 252 may use, for example, at least one of a capacitive type, a resistive type, an infrared type, and an ultrasonic type. Furthermore, the touch panel 252 may further include a control circuit. The touch panel 252 may further include a tactile layer to provide a tactile reaction to a user. The (digital) pen sensor 254 may include, for example, a recognition sheet that is a part of, or separate from, the touch panel. The key 256 may include, for example, a physical button, an optical key, or a keypad. The ultrasonic input device 258 may detect ultrasonic waves, which are generated by an input tool, through a microphone (for example, a microphone 288) to identify data corresponding to the detected ultrasonic waves.

The display 260 (for example, the display 160) may include a panel 262, a hologram device 264, a projector 266, and/or a control circuit for controlling the same. The panel 262 may be implemented to be, for example, flexible, transparent, or wearable. The panel 262, together with the touch panel 252, may be configured as one or more modules. According to an embodiment, the panel 262 may include a pressure sensor (or a POS sensor) which may measure the strength of pressure of a user's touch. The pressure sensor may be implemented so as to be integrated with the touch panel 252, or may be implemented as one or more sensors separate from the touch panel 252. The hologram device 264 may show a three-dimensional image in the air using light interference. The projector 266 may display an image by projecting light onto a screen. The screen may be located, for example, in the interior of, or on the exterior of, the electronic device 201. The interface 270 may include, for example, an HDMI 272, a USB 274, an optical interface 276, or a D-subminiature (D-sub) interface 278. The interface 270 may be included in, for example, the communication circuit 170 illustrated in FIG. 1. Additionally or alternatively, the interface 270 may, for example, include a Mobile High-definition Link (MHL) interface, a Secure Digital (SD) card/Multimedia Card (MMC) interface, or an Infrared Data Association (IrDA) standard interface.

The audio module 280 may bidirectionally convert, for example, sound and electrical signals. At least some elements of the audio module 280 may be included, for example, in the input/output interface 150 illustrated in FIG. 1. The audio module 280 may process sound information that is input or output through, for example, a speaker 282, a receiver 284, earphones 286, the microphone 288, and the like. The camera module 291 is, in some embodiments, a device that can photograph a still image and a moving image. According to an embodiment, the camera module 291 may include one or more image sensors (for example, a front sensor or a rear sensor), a lens, an image signal processor (ISP), or a flash (for example, an LED or xenon lamp). The power management module 295 may manage, for example, the power of the electronic device 201. According to an embodiment, the power management module 295 may include a power management integrated circuit (PMIC), a charger IC, or a battery or fuel gauge. The PMIC may use a wired and/or wireless charging method. Examples of the wireless charging method may include a magnetic resonance method, a magnetic induction method, an electromagnetic wave method, and the like. Additional circuits (for example, a coil loop, a resonance circuit, a rectifier, and the like) for wireless charging may be further included. The battery gauge may measure, for example, the remaining charge of the battery 296 and a voltage, current, or temperature while charging. The battery 296 may include, for example, a rechargeable battery and/or a solar battery.

The indicator 297 may display a particular state, for example, a booting state, a message state, a charging state, or the like of the electronic device 201 or a part (for example, the processor 210) of the electronic device 201. The motor 298 may convert an electrical signal into a mechanical vibration and may generate a vibration, a haptic effect, or the like. The electronic device 201 may include a mobile TV support device that can process media data according to a standard such as digital multimedia broadcasting (DMB), digital video broadcasting (DVB), MEDIAFLO™, and the like. Each of the above-described component elements of hardware according to the present disclosure may be configured with one or more components, and the names of the corresponding component elements may vary based on the type of electronic device. According to various embodiments, the electronic device (for example, the electronic device 201) may not include some elements, or may further include additional elements. Some of the elements may be coupled to constitute one object, but the electronic device may perform the same functions as those of the corresponding elements before being coupled to each other.

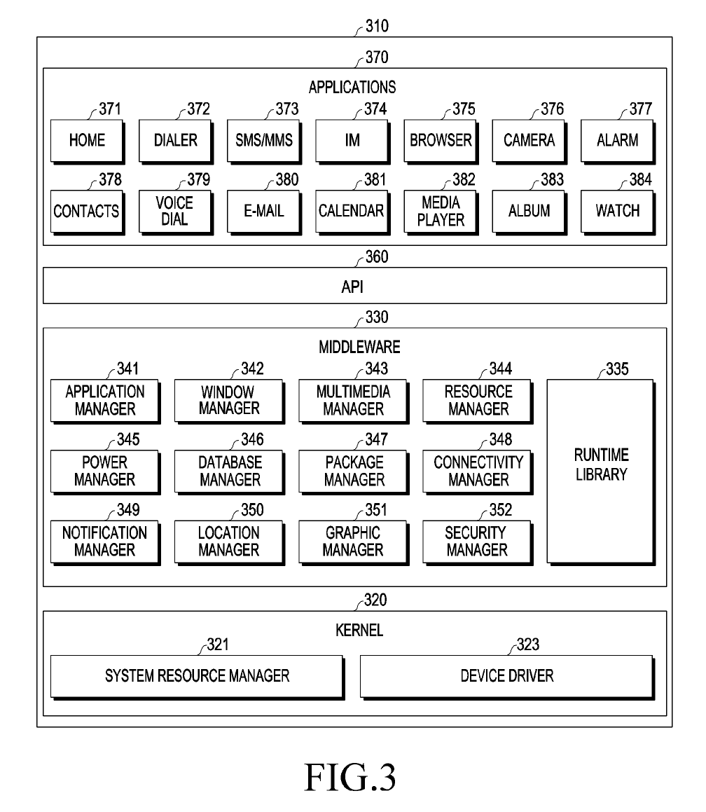

FIG. 3 illustrates, in block diagram format, a program module according to various embodiments. According to an embodiment, the program module 310 (for example, the program 140) may include an Operating System (OS) for controlling resources related to the electronic device (for example, the electronic device 101) and/or various applications (for example, the applications 147) executed in the operating system. The operating system may include, for example, ANDROID™, IOS™, WINDOWS™, SYMBIAN™, TIZEN™, OR BADA™. Referring to the non-limiting example of FIG. 3, the program module 310 may include a kernel 320 (for example, the kernel 141), middleware 330 (for example, the middleware 143), an API 360 (for example, the API 145), and/or applications 370 (for example, the applications 147). At least some of the program module 310 may be preloaded on the electronic device, or may be downloaded from an external electronic device (for example, the electronic device 102 or 104, or the server 106).

The kernel 320 may include, for example, a system resource manager 321 and/or a device driver 323. The system resource manager 321 may control, allocate, or retrieve system resources. According to an exemplary embodiment, the system resource manager 321 may include a process manager, a memory manager, a file-system manager, or the like. The device driver 323 may include, for example, a display driver, a camera driver, a Bluetooth driver, a shared memory driver, a USB driver, a keypad driver, a Wi-Fi driver, an audio driver, or an Inter-Process Communication (IPC) driver. The middleware 330 may provide, for example, a function required by the applications 370 in common, or may provide various functions to the applications 370 through the API 360 such that the applications 370 can efficiently use the limited system resources within the electronic device. According to an embodiment, the middleware 330 may include at least one of a runtime library 335, an application manager 341, a window manager 342, a multimedia manager 343, a resource manager 344, a power manager 345, a database manager 346, a package manager 347, a connectivity manager 348, a notification manager 349, a location manager 350, a graphic manager 351, and a security manager 352.

The runtime library 335 may include, for example, a library module that a compiler uses in order to add a new function through a programming language while the applications 370 are being executed. The runtime library 335 may manage input/output, manage memory, or process arithmetic functions. The application manager 341 may manage, for example, the life cycles of the applications 370. The window manager 342 may manage GUI resources used for a screen. The multimedia manager 343 may identify formats required for reproducing various media files, and may encode or decode a media file using a codec suitable for the corresponding format. The resource manager 344 may manage the source code of the applications 370 or the space in memory. The power manager 345 may manage, for example, the capacity or power of a battery and may provide power information required for operating the electronic device. According to an embodiment, the power manager 345 may operate in conjunction with a Basic Input/Output System (BIOS). The database manager 346 may, for example, generate, search, or change databases to be used by the applications 370. The package manager 347 may manage the installation or update of an application that is distributed in the form of a package file.

The connectivity manager 348 may manage, for example, a wireless connection. The notification manager 349 may provide information on an event (for example, an arrival message, an appointment, a proximity notification, or the like) to a user. The location manager 350 may manage, for example, the location information of the electronic device. The graphic manager 351 may manage a graphic effect to be provided to a user and a user interface relating to the graphic effect. The security manager 352 may provide, for example, system security or user authentication. According to an embodiment, the middleware 330 may include a telephony manager for managing a voice or video call function of the electronic device or a middleware module that is capable of forming a combination of the functions of the above-described elements. According to an embodiment, the middleware 330 may provide a module specified for each type of OS. Furthermore, the middleware 330 may dynamically remove some existing elements, or may add new elements. The API 360 is, for example, a set of API programming functions, and may be provided with different configurations according to operating systems. For example, in the case of Android or iOS, one API set may be provided for each platform, and in the case of Tizen, two or more API sets may be provided for each platform.

The applications 370 may include, for example, a home application 371, a dialer application 372, an SMS/MMS application 373, an instant messaging (IM) application 374, a browser application 375, a camera application 376, an alarm application 377, a contact application 378, a voice dial application 379, an email application 380, a calendar application 381, a media player application 382, an album application 383, a watch application 384, a health-care application (for example, for measuring exercise quantity or blood glucose), or an application providing environmental information (for example, atmospheric pressure, humidity, or temperature information). According to an embodiment, the applications 370 may include an information exchange application that can support the exchange of information between the electronic device and an external electronic device. The information exchange application may include, for example, a notification relay application for relaying particular information to an external electronic device or a device management application for managing an external electronic device. For example, the notification relay application may relay notification information generated in the other applications of the electronic device to an external electronic device, or may receive notification information from an external electronic device and provide the received notification information to a user. The device management application may install, delete, or update the functions (for example, turning on/off the external electronic device itself (or some elements thereof) or adjusting the brightness (or resolution) of a display) of an external electronic device that communicates with the electronic device or applications executed in the external electronic device. According to an embodiment, the applications 370 may include applications (for example, a health care application of a mobile medical appliance) that are designated according to the attributes of an external electronic device. According to an embodiment, the applications 370 may include applications received from an external electronic device. At least some of the program module 310 may be implemented (for example, executed) by software, firmware, hardware (for example, the processor 210), or a combination of two or more thereof, and may include a module, a program, a routine, an instruction set, or a process for performing one or more functions.

The term “module” as used herein may include a unit consisting of hardware, software, or firmware, and may, for example, be used interchangeably with the term “logic”, “logical block”, “component”, “circuit”, or the like. The “module” may be an integrated component or a minimum unit for performing one or more functions or a part thereof. The “module” may be mechanically or electronically implemented, and may include, for example, an Application-Specific Integrated Circuit (ASIC) chip, a Field-Programmable Gate Array (FPGA), or a programmable logic device, which is currently known or is to be developed in the future, for performing certain operations.

At least some of devices (for example, modules or functions thereof) or methods (for example, operations) according to various embodiments may be implemented by an instruction which is stored in a computer-readable storage medium (for example, the memory 130) in the form of a program module. The instruction, when executed by a processor (for example, the processor 120), may cause the one or more processors to execute the function corresponding to the instruction. The computer-readable storage medium may include a hard disk, a floppy disk, magnetic media (for example, a magnetic tape), optical media (for example, CD-ROM, DVD), magneto-optical media (for example, a floptical disk), internal memory, and the like. The instruction may include code made by a complier or code that can be executed by an interpreter. The programming module according to the present disclosure may include one or more of the aforementioned elements or may further include other additional elements, or some of the aforementioned elements may be omitted. Operations performed by a module, a programming module, or other elements according to various embodiments may be executed sequentially, in parallel, repeatedly, or in a heuristic manner. At least some operations may be executed according to another sequence, may be omitted, or may further include other operations.

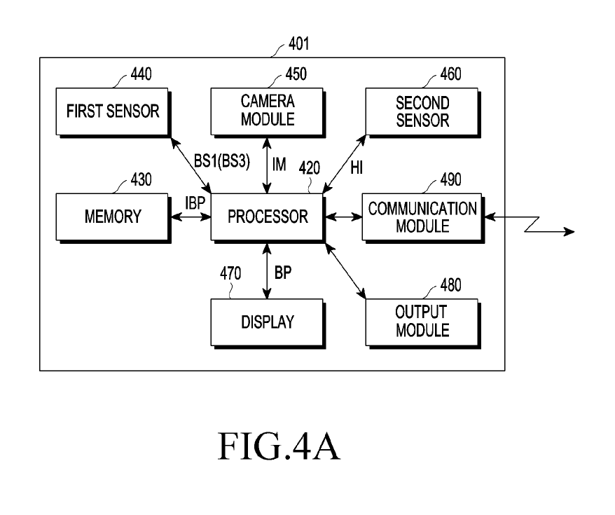

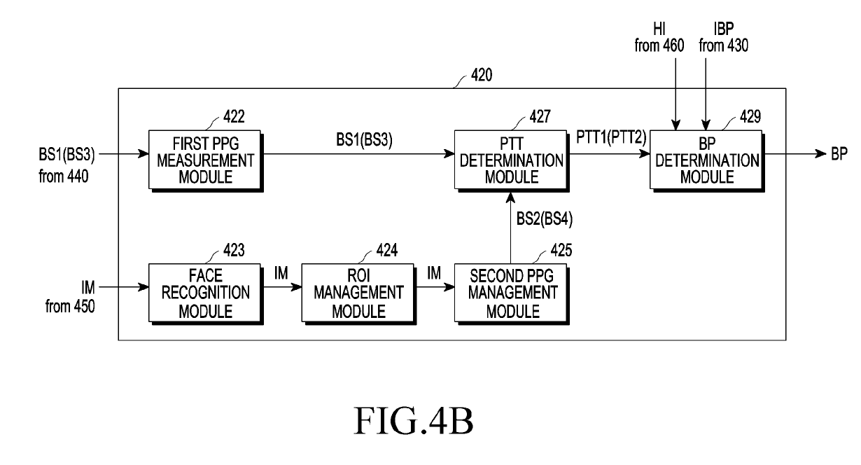

FIG. 4A illustrates, in block diagram format, an electronic device according to various embodiments.

Referring to the non-limiting example of FIG. 4A, an electronic device 401 (for example, the electronic device 101 or 201) may include a processor 420 (for example, the processor 120 or 210), a memory 430 (for example, the memory 130 or 230), a first sensor 440 (for example, the biometric sensor 240I), a camera module 450 (for example, the camera module 291), a second sensor 460 (for example, the acceleration sensor 240E), a display 470 (for example, the display 160 or 260), an output device 480 (for example, the speaker 282, the indicator 297, and/or the motor 298), and a communication module 490 (for example, the communication module 220).

Embodiments of electronic device 401 may be implemented to be substantially the same or similar to the electronic device (the electronic device 101 or 201) described with reference to FIGS. 1 and 2.

The processor 420 may control the overall operation of the electronic device 401.

The processor 420 may acquire a biometric signal of the user through the first sensor 440 and/or the camera module 450. Further, the processor 420 may measure biometric information of the user (for example, a heart rate, oxygen saturation, stress, and blood pressure of the user) based on the acquired biometric signal.

According to an embodiment, the processor 420 may acquire a first biometric signal (BS1) of the user through the first sensor 440, and may acquire a second biometric signal (BS2, see FIG. 4B) of the user through the camera module 450 at a first location.

The processor 420 may acquire a third biometric signal (BS3) of the user through the first sensor 440, and may acquire a fourth biometric signal (BS4, see FIG. 4B) of the user through the camera module 450 at a second location. For example, the first location and the second location may be locations having different heights.

For example, the first biometric signal (BS1) and the second biometric signal (BS2) may be biometric signals acquired at the first location having a first height. The third biometric signal (BS3) and the fourth biometric signal (BS4) may be biometric signals acquired at the second location having a second height.

The first biometric signal (BS1) and the third biometric signal (BS3) may be acquired at the same body position. For example, the first biometric signal (BS1) and the third biometric signal (BS3) may be acquired at a user's fingers.

The second biometric signal (BS2) and the fourth biometric signal (BS4) may be measured at the same body position. For example, the second biometric signal (BS2) and the fourth biometric signal (BS4) may be acquired at a region of interest (for example, a region under eyes) on the user's face.

The first, biometric signal (BS1), the second biometric signal (BS2), the third biometric signal (BS3), and the fourth biometric signal (BS4) may include a photoplethysmography (PPG) signal of the user.

The processor 420 may determine a Blood Pressure (BP) based on the first biometric signal (BS1) and the second biometric signal (BS2), which are acquired at the first location, and the third biometric signal (BS3) and the fourth biometric signal (BS4), which are acquired at the second location.

The processor 420 may acquire first biometric information based on the difference between the first biometric signal (BS1) and the second biometric signal (BS2). The processor 420 may acquire second biometric information based on the difference between the third biometric signal (BS3) and the fourth biometric signal (BS4). The processor 420 may acquire information related to blood pressure based on the first biometric information and the second biometric information. For example, the first biometric information and the second biometric information may include information on a Pulse Transit Time (PTT).

Hereinafter, for convenience of description, it is assumed that the first biometric information is a first PTT and that the second biometric information is a second PTT. However, embodiments according to the present disclosure are not limited thereto.

The processor 420 may compare at least one peak point included in the first biometric signal (BS1) and the second biometric signal (BS2). For example, the processor 420 may compare the peak point of the first biometric signal (BS1) and the peak point of the second biometric signal (BS2) received subsequently (continuously) to the first biometric signal (BS1). Further, the processor 420 may determine an average value or an intermediate value of at least one comparison value as a first PTT.

Similarly, the processor 420 may compare at least one peak point included in the third biometric signal (BS3) and the fourth biometric signal (BS4). For example, the processor 420 may compare the peak point of the third biometric signal (BS2) and the peak point of the fourth biometric signal (BS4) received subsequently (continuously) to the third biometric signal (BS3). Further, the processor 420 may determine an average value or an intermediate value of at least one comparison value as a second PTT.

The processor 420 may determine the blood pressure of the user based on the first PTT and the second PTT. For example, the processor 420 may determine the first PTT at the first location and the second PTT at the second location, and compare the first PTT and the second PTT. The processor 420 may determine the difference between the first PTT and the second PTT and detect the blood pressure of the user based on the difference.

According to an embodiment, the first location and the second location may be locations having different heights. For example, the user may place the first electronic device at the first location having the first height and then move the electronic device to be placed at the second location having the second height. For example, the first location may be a location corresponding to the user's head, and the second location may be a location corresponding to the user's chest.

The processor 420 may determine the difference between the first location and the second location through the second sensor 460. The processor 420 may determine the accurate blood pressure (BP) based on the determined height difference. The processor 420 may determine the accurate blood pressure (BP) based on the biometric information measured at different heights.

The processor 420 may determine the accurate blood pressure (BP) based on the Initial Blood Pressure (IBP) stored in the memory 430. For example, the initial blood pressure (IBP) may be used as an offset value for determining the user's blood pressure based on the PTT.

According to an embodiment, the initial blood pressure (IBP) may be automatically set by the processor 420 or manually set by the user. For example, the initial blood pressure (IBP) may be a blood pressure measured through a medical device such as a blood pressure gauge. At this time, the initial blood pressure (IBP) may be directly input to the electronic device 401 by the user, or may be acquired from a server or another electronic device through the communication module 490. Meanwhile, the processor 420 may automatically set the initial blood pressure (IBP) based on user's personal data (for example, previously measured blood pressure, gender, age, and weight).

The processor 420 may display the determined blood pressure (BP) on the display 470. Further, the processor 420 may store the determined blood pressure (BP) in the memory 430.

The processor 420 may transmit the determined blood pressure (BP) to another electronic device through the communication module 490.

Meanwhile, the processor 420 may provide the same clock to the first sensor 440 and the camera module 450. The processor 420 may acquire the first biometric signal (BS1) and the second biometric signal (BS2) at the first location based on the same clock. Further, the processor 420 may acquire the third biometric signal (BS3) and the fourth biometric signal (BS4) at the second location based on the same clock. For example, the processor 420 may synchronize the first sensor 440 and the camera module 450 based on the same clock and operate them.

The memory 430 may store the blood pressure (BP) measured by the processor 420. Further, the memory 430 may store the initial blood pressure (IBP). The memory 430 may store the biometric signals acquired through the first sensor 440 and the camera module 450. The memory 430 may store data on the user's body (for example, gender, age, weight, blood type, and/or health state). For example, the memory 440 may be implemented as nonvolatile memory or volatile memory.

The first sensor 440 may acquire the biometric signal (BS1 and/or BS3) of the user. The first sensor 440 may transmit the acquired biometric signal to the processor 420. For example, the first sensor 440 may be implemented as an optical sensor and/or a photoplethysmogram (PPG) sensor.

The first sensor 440 may include a light emitter (not shown) and a light receiver (not shown). For example, the light emitter may output a light (or a light signal) to the user's skin. For example, the light emitter may output at least one of an infrared ray and a red, green, and/or blue light (or optical signal). Further, the light emitter may include at least one module for outputting an infrared ray and a red, green, and/or blue light.

The light receiver may receive at least some of the light (or optical signal) reflected by the user's body tissue (for example, skin, skin tissue, layer of fat, vein, artery, and/or capillary) among the light (or optical signal) output from the light emitter. Further, the light receiver may output the biometric signal (BS1 and/or BS3) corresponding to the received light. For example, the light receiver may include a photo diode.

The biometric signals (BS1 and BS3) may be signals reflected by the user's skin (or user's skin tissue) among the light (or optical signal) output through the first sensor 440. For example, the biometric signals (BS1 and BS3) may be signals reflected by the user's skin (or user's skin tissue) and receive through the light receiver of the first sensor 440. For example, the biometric signals (BS1 and BS3) may include a PPG signal.

The camera module 450 may photograph a subject and generate an image (IM). The camera module 450 may transmit the image (IM) to the processor 420. The camera module 450 may photograph the image (IM) having a predetermined number of frames (or predetermined frame rates) per second. For example, the camera module 450 may photograph the image (IM) having a frame rate of 30 frames per second (fps).

The camera module 450 may include at least one camera selected from among an infrared camera, an RGB camera, and an iris recognition camera.

According to an embodiment, the camera module 450 may generate the image (IM) by photographing the user's body part (for example, the part of the user's face). For example, the processor 420 may acquire the second biometric signal (BS2) and/or the fourth biometric signal (BS4) based on a change in the user's body part included in the image (IM). For example, the processor 420 may acquire the second biometric signal (BS2) and/or the fourth biometric signal (BS4) based on the change in the user's body part included in a plurality of images (IM) photographed during a predetermined time.

The second sensor 460 may generate a signal corresponding to a user's motion. Further, the second sensor 460 may transmit the generated signal to the processor 420. For example, the second sensor 460 may generate a signal for an acceleration value (and/or an angular speed value) corresponding to the user's motion. Meanwhile, the second sensor 460 may include at least one of an acceleration sensor (the acceleration sensor 240e of FIG. 2), an angular sensor, and a gyro sensor (for example, the gyro sensor 240b of FIG. 2).

According to an embodiment, the second sensor 460 may generate information on a height difference (Height Information (HI)) corresponding to acceleration between the first location and the second location based on the user's motion. The processor 420 may determine a height difference between the first location and the second location through the information on the height difference (HI).

The display 470 may display the blood pressure (BP) measured by the processor 420. For example, the display 470 may be implemented as a touch screen.

The display 470 (for example, a touch screen) may receive input for measuring the user's blood pressure. Further, the display 470 (for example, a touch screen) may transmit a signal corresponding to the received input (for example, a signal corresponding to the input for measuring the blood pressure) to the processor 420.

The output device 480 may inform the user of the state of the measured user's blood pressure. For example, the output device 480 may inform the user of the state of the measured blood pressure through auditory, tactile, and visual means.

The communication module 490 may transmit the blood pressure (BP) measured by the processor 420. Further, the communication module 450 may receive a biometric signal and/or biometric information (for example, user's blood pressure) measured by an external electronic device.

FIG. 4B illustrates, in block diagram format, operations of a processor according to embodiments of this disclosure, for example processor 420 illustrated in FIG. 4A.

Referring to the non-limiting example of FIG. 4B, the processor 420 may include a first PPG measurement module 422, a face recognition module 423, a Region Of Interest (ROI) management module 424, a second PPG measurement module 425, a PTT determination module 427, and a BP determination module 429.

The first PPG measurement module 422 may receive a first biometric signal (BS1) and a third biometric signal (BS3). For example, the first biometric signal (BS1) or the third biometric signal (BS3) may be a PPG signal for the user.

The first PPG measurement module 422 may acquire a first PPG signal (BS1) corresponding to the first biometric signal (BS1) at a first location. Further, the first PPG measurement module 422 may acquire a third PPG signal (BS3) corresponding to the third biometric signal (BS3) at a second location. In addition, the first PPG measurement module 422 may remove noise of the first PPG signal (BS1) and/or the third PPG signal (BS3).

According to an embodiment, the first PPG measurement module 422 may acquire the first PPG signal (BS1) and/or the third PPG signal (BS3) having a first frequency. For example, the first frequency may be 100 Hz.

The first PPG measurement module 422 may transmit the first PPG signal (BS1), measured at the first location, to the PTT determination module 427. Further, the second PPG measurement module 422 may transmit the third PPG signal (BS3), measured at the second location, to the PTT determination module 427.

In order to acquire the PPG signal from the user's face included in the image (IM), the face recognition module 423 may recognize the face included in the image (IM). The face recognition module 423 may determine feature points of the face included in the image (IM) and recognize the face (or face region) included in the image (IM) based on the determined feature points. Meanwhile, information on the feature points of the face may be stored in a security zone of the memory 430.

The face recognition module 423 may transmit the image (IM) including the recognized face (or face region) to the ROI management module 424.

The face recognition module 423 may determine whether the user is a registered user based on the feature points of the face included in the image (IM). For example, when the face included in the image (IM) is a registered user's face, the face recognition module 423 may measure the blood pressure. On the other hand, when the face included in the image (IM) is not a registered user's face, the face recognition module 423 may stop measuring the blood pressure.

The ROI management module 424 may manage a Region of Interest (ROI) of the face included in the image (IM) in order to acquire the PPG signal.

The ROI management module 424 may determine an ROI of the face included in the image (IM) from which the PPG signal can be easily acquired. For example, the ROI management module 424 may determine a region of the face having thin skin (for example, the region under the eyes) as the ROI. Further, the ROI management module 424 may determine the ROI according to the position and/or the direction of the face included in the image (IM). For example, information on the ROI may be stored in the memory 430.

The second PPG measurement module 425 may acquire a second biometric signal (BS2) from the image (IM) photographed at the first location. The second PPG measurement module 425 may acquire a fourth biometric signal (BS4) from the image (IM) photographed at the second location. For example, the second PPG measurement module 425 may acquire the second biometric signal (BS2) and/or the fourth biometric signal (BS4) based on a change in the ROI included in the image (IM).

The second PPG measurement module 425 may acquire a second PPG signal (BS2) corresponding to the second biometric signal (BS2) at the first location. Further, the second PPG measurement module 425 may acquire a fourth PPG signal (BS4) corresponding to the fourth biometric signal (BS4) at the second location. In addition, the second PPG measurement module 425 may remove noise of the second PPG signal (BS2) or the fourth PPG signal (BS4).

According to an embodiment, the second PPG measurement module 425 may acquire the second PPG signal (BS2) or the fourth PPG signal (BS4) having a second frequency. For example, the second frequency may be 30 Hz.

According to an embodiment, the second PPG measurement module 425 may interpolate the second PPG signal (BS2) having the second frequency to fit the first frequency. That is, the second PPG measurement module 425 may interpolate the PPG signal (BS2 and/or BS4) to fit the frequency which is the same as that of the first PPG signal (BS1) or the third PPG signal (BS3) output from the first PPG measurement module 422. For example, the second PPG measurement module 425 may interpolate the second PPG signal (BS2) or the fourth PPG signal (BS4) having 30 Hz to fit 100 Hz.

The second PPG measurement module 425 may transmit the interpolated second PPG signal (BS2) to the PTT determination module 427. Further, the second PPG measurement module 425 may transmit the interpolated fourth PPG signal (BS4) to the PTT determination module 427.

Although in the non-limiting example of FIG. 4B the second PPG measurement module 425 is separated from the first PPG measurement module 422, according to other embodiments, the first PPG measurement module 422 and the second PPG measurement module 425 may be implemented as a single measurement module.

The PTT determination module 427 may acquire a first PTT (PTT1) for the first location based on the difference between the first PPG signal (BS1) and the second PPG signal (BS2). For example, the PTT determination module 427 may compare a peak point of the first PPT signal (BS1) and a peak point of the second PPT signal (BS2), received subsequently to the first PPT signal (BS1), and acquire the first PTT (PTT1) according to the comparison result.

The PTT determination module 427 may acquire a second PTT (PTT2) for the second location based on the difference between the third PPT signal (BS3) and the fourth PPT signal (BS4). For example, the PTT determination module 427 may compare a peak point of the third PPT signal (BS3) and a peak point of the fourth PPT signal (BS4), received subsequently to the third PPT signal (BS3), and acquire the second PTT (PTT2) according to the comparison result.

The PTT determination module 427 may sequentially acquire the first PTT (PTT1) for the first location and the second PTT (PTT2) for the second location. For example, the PTT determination module 427 may first acquire the first PTT (PTT1) for the first location and then acquire the second PTT (PTT2) for the second location.

The PTT determination module 427 may transmit the first PTT (PTT1) for the first location to the BP determination module 429. Further, the PTT determination module 427 may transmit the second PTT (PTT2) for the second location to the BP determination module 429.

The BP determination module 429 may determine the blood pressure (BP) based on the first PTT (PTT1) acquired at the first location and the second PTT (PTT2) acquired at the second location. For example, the BP determination module 429 may determine the blood pressure (BP) based on the first PTT (PTT1) for the first location, the second PTT (PTT2) for the second location, both of which are measured by the PTT determination module, the initial blood pressure (IBP) stored in the memory 430, and the difference between the first location and the second location of the electronic device 401, which is acquired through the second sensor 460.

According to an embodiment, the BP determination module 429 may determine a change in the user's blood pressure based on the first PTT (PTT1). The BP determination module 429 may calibrate the change in the blood pressure according to the time based on the initial blood pressure (IBP), the height difference between the first location and the second location, and the difference between the first PTT (PTT1) and the second PTT (PTT2). The BP determination module 429 may determine the user's blood pressure based on the calibrated change in the blood pressure according to the time.

The BP determination module 429 may acquire the initial blood pressure (IBP) stored in the memory 430. Further, the BP determination module 429 may acquire the initial blood pressure (IBP) from an external electronic device.

For example, the BP determination module 429 may determine the blood pressure (BP) based on Equation (1).

BP=A*F(PTT1)+B Equation (1)

In Equation (1), A denotes ΔBP/|PTT1−PTT2|, F(PTT1) denotes a function for PTT1 (for example, a change in the blood pressure according to the time), and B denotes an initial blood pressure (IBP). For example, F(PTT1) may be a function linearly proportional to PTT1. Meanwhile, ΔBP may be calculated using Equation (2).

ΔBP=p*g*h Equation (2)

In Equation (2), p denotes a specific gravity of the blood, g denotes the acceleration of gravity, and h denotes a height difference between a first location and a second location. For example, p and g may be constants.

According to an embodiment, the BP determination module 429 may acquire information (HI) on the height difference between the first location and the second location corresponding to the acceleration of the electronic device 401, which has moved from the first location to the second location, through the second sensor 460. The BP determination module 429 may determine the height difference (h) between the first location and the second location by analyzing the information (HI) on the height difference. The BP determination module 429 may determine ΔBP based on Equation (2).

For example, the BP determination module 429 may calibrate an inclination of the change in the blood pressure according to the time with respect to the first PTT (PTT1) through the difference (|PTT1−PTT2|) between the first PTT (PTT1) and the second PTT (PTT2), and may calibrate the initial blood pressure (IBP), which is an offset, through the change in the blood pressure according to the time with respect to the first PTT (PTT1). The BP determination module 429 may determine the user's blood pressure based on the calibrated change in the blood pressure according to the time.

Accordingly, the BP determination module 429 may determine the blood pressure (BP) based on the first PTT (PTT1), the second PTT (PTT2), the initial blood pressure (IBP), and the height difference (h) between the first location and the second location.

The BP determination module 429 may display the determined blood pressure (BP) on the display 470. Further, the BP determination module 429 may store the determined blood pressure (BP) in the memory 430.

Although FIG. 4B illustrates an embodiment in which the first PPG measurement module 422, the face recognition module 423, the ROI management module 424, the second PPG measurement module 425, the PTT determination module 427, and the BP determination module 429 are separated from each other, the modules may also be implemented to be integrated into one or more modules.

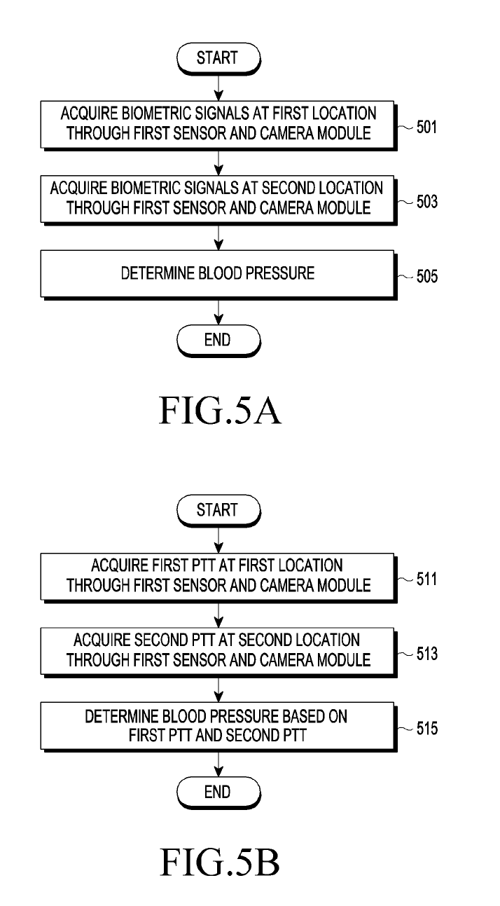

FIGS. 5A and 5B illustrate operations of a method of an electronic device according to various embodiments.

Referring to the non-limiting example of FIG. 5A, the processor 420 (for example, the processor 420 of FIG. 4A) may acquire the first biometric signal (BS1) and the second biometric signal (BS2) through the first sensor 440 and the camera module 450 at the first location in step 501. For example, the first biometric signal (BS1) and the second biometric signal (BS2) may include a PPG signal.

The processor 420 may acquire the third biometric signal (BS3) and the fourth biometric signal (BS4) through the first sensor 440 and the camera module 450 at the second location in step 503. For example, the third biometric signal (BS3) and the fourth biometric signal (BS4) may include a PPG signal.

The processor 420 may determine the blood pressure based on the first biometric signal (BS1) and the second biometric signal (BS2) acquired at the first location and the third biometric signal (BS3) and the fourth biometric signal (BS4) acquired at the second location in step 505.

Referring to the non-limiting example of FIG. 5B, the processor 420 (for example, the processor 420 of FIG. 4A) may acquire the first biometric signal (BS1) and the second biometric signal (BS2) through the first sensor 440 and the camera module 450 at the first location.

The processor 420 may acquire the first PTT (PTT1) for the first location based on the first biometric signal (BS1) and the second biometric signal (BS2) acquired through the first sensor 440 and the camera module 450 in step 511.

The processor 420 may acquire the third biometric signal (BS3) and the fourth biometric signal (BS4) at the second location through the first sensor 440 and the camera module 450.

The processor 420 may acquire the second PTT (PTT2) for the second location based on the third biometric signal (BS3) and the fourth biometric signal (BS4) acquired through the first sensor 440 and the camera module 450 in step 513.

The processor 420 may determine the blood pressure (BP) based on the first PTT (PTT1) for the first location and the second PTT (PTT2) for the second location. According to an embodiment, the processor 420 may determine the blood pressure (BP) based on the first PTT (PTT1), the second PTT (PTT2), the initial blood pressure (IBP), and the height difference between the first location and the second location.

Meanwhile, hereinafter, it is assumed that the biometric signals (BS1 to BS4) are PPG signals for convenience of description. However, the technical idea of the present disclosure is not limited thereto.

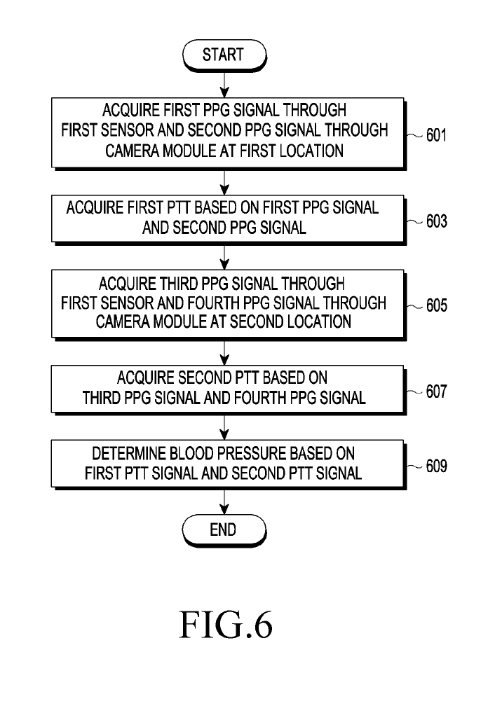

FIG. 6 illustrates operations of an electronic device according to various embodiments.

Referring to the non-limiting example of FIG. 6, the processor 420 (for example, the processor 420 of FIG. 4A) may acquire the first PPG signal (BS1) through the first sensor 440 and the second PPG signal (BS2) through the camera module 450 at the first location in step 601.

For example, at the first location (for example, the height corresponding to the user's head), the processor 420 may acquire the first PPG signal (BS1) from a part of the user's body (for example, fingers) through the first sensor 440 and acquire the second PPG signal (BS2) from another part of the user's body (for example, an ROI of the face) through the camera module 450.

The processor 420 may acquire the first PTT (PTT1) for the first location based on the first PPG signal (BS1) and the second PPG signal (BS2) in step 603.

The processor 420 may acquire the third PPG signal (BS3) through the first sensor 440 and the fourth PPG signal (BS4) through the camera module 450 at the second location in step 605.

For example, at the second location (for example, a height corresponding to the user's chest or waist), the processor 420 may acquire the third PPG signal (BS3) from a part of the user's body (for example, the fingers) through the first sensor 440, and may acquire the fourth PPG signal (BS4) from another part of the user's body (for example, an ROI of the face) through the camera module 450.

The processor 420 may acquire the second PTT (PTT2) for the second location based on the third PPG signal (BS3) and the fourth PPG signal (BS4) in step 607.

The processor 420 may determine the blood pressure based on the first PTT (PTT1) and the second PTT (PTT2) in step 609. According to an embodiment, the processor 420 may determine a more accurate blood pressure (BP) based on the first PTT (PTT1), the second PTT (PTT2), the initial blood pressure (IBP), and the height difference between the first location and the second location.

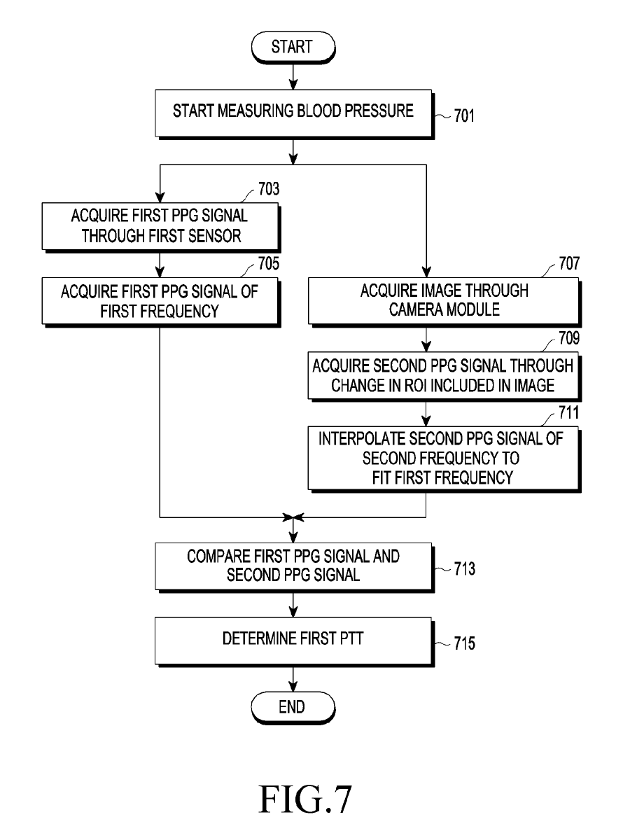

FIG. 7 illustrates operations of an e electronic device according to various embodiments.

Referring to the non-limiting example of FIG. 7, the processor 420 (for example, the processor 420 of FIG. 4A) may start measuring the blood pressure in response to a request for measuring the blood pressure in step 701. For example, when a user's request (for example, input corresponding to the request for measuring the blood pressure) is detected, the processor 420 may start the operation of measuring the user's blood pressure.

The processor 420 may acquire the first PPG signal (BS1) through the first sensor 440 at the first location in step 703. For example, when the electronic device 401 is positioned at the first location (for example, the height corresponding to the user's head) having the first height, the processor 420 may acquire the first PPG signal (BS1) from the part of the user's body (for example, fingers) through the first sensor 440.

The processor 420 may acquire the first PPG signal (BS1) of the first frequency in step 705. For example, the first frequency may be 100 Hz.

At the first location, the processor 420 may acquire a plurality of images (IMS) having a predetermined frame rate through the camera module 450 in step 707. For example, the predetermined frame rate may be 30 frames per second (fps).

The processor 420 may acquire the second PPG signal (BS2) through a change in the ROI (for example, the region of the face under the eyes) included in the plurality of images (IM) in step 709. For example, the processor 420 may analyze the change (for example, a change in a color of the ROI) in the ROI included in each of the plurality of images (IM) and acquire the second PPG signal (BS2) of the second frequency based on the change in the ROI in step 709. For example, the second frequency may be 30 Hz.

In order to match the first frequency and the second frequency, the processor 420 may interpolate the second PPG signal (BS2) of the second frequency to fit the first frequency in step 711. For example, in order to compare the first PPG signal (BS1) and the second PPG signal (BS2), the processor 420 may interpolate the first PPG signal (BS1) and the second PPG signal (BS2) to fit the same frequency. For example, the processor 420 may interpolate the second PPG signal (BS2) of 30 Hz to fit 100 Hz. Meanwhile, when the first frequency and the second frequency are the same as each other, the processor 420 may not interpolate the second PPG signal (BS2) of the second frequency.

The processor 420 may simultaneously or sequentially acquire the first PPG signal (BS1) and the second PPG signal (BS2).

Since the first PPG signal (BS1) and the second PPG signal (BS2) are measured at different positions of the body, they may be different from each other. For example, the first PPG signal (BS1) may be a signal for the part of the user's body (for example, fingers) and the second PPG signal (BS2) may be a signal for the other part of the user's body (for example, ROI of the face).

The processor 420 may compare the first PPG signal (BS1) and the second PPG signal (BS2) in step 713. For example, in order to determine the difference between the first PPG signals (BS1) and the second PPG signal (BS2), the processor 420 may compare peak points, lowest points, and/or greatest change points of the first PPG signal (BS1) and the second PPG signal (BS2).

The processor 420 may acquire the first PTT (PTT1) based on the difference between the first PPG signal (BS1) and the second PPG signal (BS2) in step 715. For example, the processor 420 may determine, as the first PTT (PTT1), an intermediate value or an average value of at least one comparison value obtained by comparing the peak points, the lowest points, and/or the greatest change points of the first PPG signal (BS1) and the second PPG signal (BS2).

For example, the processor 420 may compare a plurality of peak points of the first PPG signal (BS1) and a plurality of peak points of the second PPG signal (BS2) and determine an intermediate value or an average of the comparison values as the first PTT (PTT1).

After acquiring the first PTT (PTT1) at the first location, the processor 420 may acquire the second PTT (PTT2) for the second location according to the above-described method (steps 701 to 715).

According to an embodiment, when the electronic device 401 moves from the first location having the first height (for example, a height corresponding to the user's head) to the second location having the second height (for example, a height corresponding to the user's chest), the processor 420 may acquire the third PPG signal (BS3) through the first sensor 440. For example, the third PPG signal (BS3) may be acquired in a body region, which is the same as that of the first PPG signal (BS1). Further, the processor 420 may acquire the fourth PPG signal (BS4) for the second location through the camera module 450. For example, the third PPG signal (BS3) may be acquired in a body region, which is the same as that of the first PPG signal (BS1). The processor 420 may compare the third PPG signal (BS3) and the fourth PPG signal (BS4) and acquire the second PTT (PTT2) for the second location (for example, a height corresponding to the user's chest) based on the difference between the third PPG signal (BS3) and the fourth PPG signal (BS4).

The processor 420 may sequentially acquire the first PTT (PTT1) for the first location (for example, a height corresponding to the user's head) and the second PTT (PTT2) for the second location (for example, a height corresponding to the user's chest).

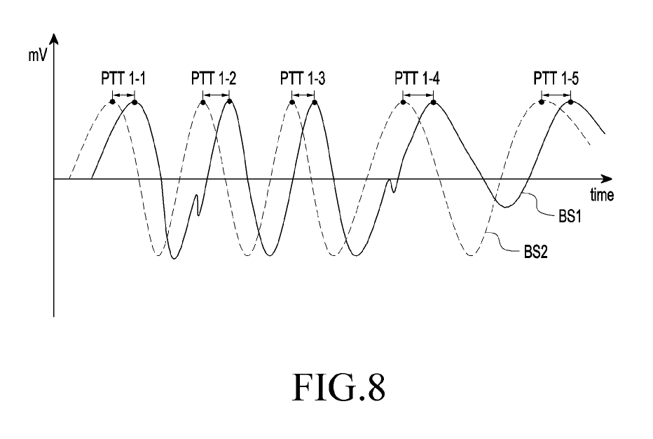

FIG. 8 is a graph illustrating aspects of the operation of acquiring first biometric information based on the first biometric signal and the second biometric signal according to various embodiments.

Referring to the non-limiting example of FIG. 8, the processor 420 (for example, the processor 420 of FIG. 4A) may acquire the first PTT (PTT1) based on a difference between the first PPG signal (BS1) and the second PPG signal (BS2).

According to an embodiment, the processor 420 may compare the first PPG signal (BS1) and the second PPG signal (BS2).

For example, the processor 420 may compare peak points of the first PPG signal (BS1) and peak points of the second PPG signal (BS2). The processor 420 may acquire comparison values (PTT1-1 to PTT1-5) according to the comparison result.

The processor 420 may determine, as the first PTT (PTT1), an intermediate value (for example, PTT1-2) of a plurality of comparison values obtained by comparing the peak points of the first PPG signal (BS1) and the second PPG signal (BS2). Further, the processor 420 may determine, as the first PTT (PTT1), an average value of the plurality of comparison values obtained by comparing the peak points of the first PPG signal (BS1) and the second PPG signal (BS2).

In addition, the processor 420 may compare lowest points or greatest change points of the first PPG signal (BS1) and the second PPG signal (BS2) and determine the first PTT (PTT1) according to the comparison result.

The processor 420 may acquire the first PTT (PTT1) for the first location according to the above-described operation method. Similarly, the processor 420 may acquire the second PTT (PTT2) for the second location according to the above-described operation method. For example, the processor 420 may sequentially acquire the first PTT (PTT1) for the first location and the second PTT (PTT2) for the second location.

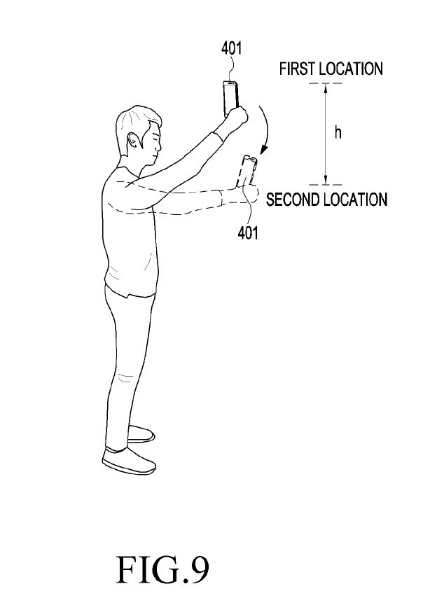

FIG. 9 illustrates aspects of acquiring biometric information at the first location and the second location according to various embodiments.

Referring to the non-limiting example of FIG. 9, the processor 420 (for example, the processor 420 of FIG. 4A) may acquire biometric signals of the user through the first sensor 440 and the camera module 450.

According to an embodiment, the processor 420 may determine the accurate blood pressure based on the first PTT (PTT1) and the second PTT (PTT2) determined through biometric signals (for example, the first PPG signal to the fourth PPG signal) acquired at different heights.

According to an embodiment, the processor 420 may acquire the first PPG signal (BS1) and the second PPG signal (BS2) through the first sensor 440 and the camera module 450 at the first location having the first height (for example, the height corresponding to the user's head). For example, when the user stretches his/her arm in an upward direction (for example, to the height corresponding to the head) while holding the electronic device 401, the processor 420 may acquire the first PPG signal (BS1) and the second PPG signal (BS2) through the first sensor 440 and the camera module 450. Further, the processor 420 may acquire the first PTT (PTT1) for the first location (for example, the height corresponding to the user's head) based on the first PPG signal (BS1) and the second PPG signal (BS2).

At the second location (for example, a height corresponding to the user's chest), the processor 420 may acquire the third PPG signal (BS3) and the fourth PPG signal (PPG4) through the first sensor 440 and the camera module 450. For example, when the user stretches his/her arm in a downward direction (for example, to the height corresponding to the chest) while holding the electronic device 401, the processor 420 may acquire the third PPG signal (BS3) and the fourth PPG signal (BS4) through the first sensor 440 and the camera module 450. Further, the processor 420 may acquire the second PTT (PTT2) for the second location (for example, the height corresponding to the user's chest) based on the third PPG signal (BS3) and the fourth PPG signal (BS4).

The processor 420 may acquire information (Height Information (HI)) on the height difference between the first location (for example, the height corresponding to the user's height) and the second location (for example, the height corresponding to the user's chest) through the second sensor 460. For example, when the electronic device 401 is moved from the first location to the second location by the user, the processor 420 may acquire information (HI) on the height difference corresponding to acceleration of the electronic device 401 moved from the first location to the second location. The processor 420 may determine a movement distance (for example, a distance between the first location and the second location) based on the acceleration included in the information (HI) on the height difference, and may determine the height difference (h) between the first location and the second location based on the movement distance. For example, the processor 420 may determine the height difference (h) between the height corresponding to the user's head and the height corresponding to the user's chest.

The processor 420 may determine the accurate blood pressure based on the height difference (h) between the first location and the second location.

Meanwhile, although FIG. 9 illustrates certain embodiments in which the electronic device 401 moves from the first location to the second location for convenience of description, the location, order, and/or direction of the movement of the electronic device 401 are not limited thereto.

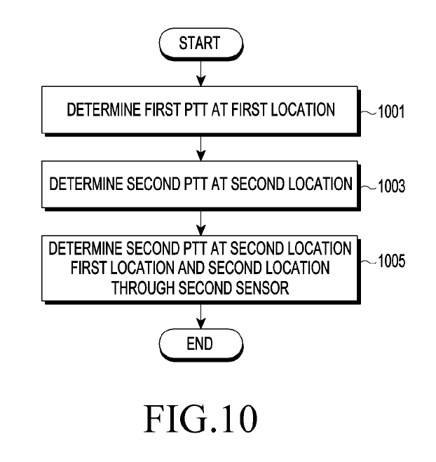

FIG. 10 is a flowchart illustrates operations of a method of determining the height difference between the first location and the second location according to various embodiments.

Referring to the non-limiting example of FIG. 10, at the first location, the processor 420 (for example, the processor 420 of FIG. 4A) may acquire the first PPG signal (BS1) and the second PPG signal (BS2) through the first sensor 440 and the camera module 450.

The processor 420 may acquire the first PTT (PTT1) for the first location based on the first PPG signal (BS1) and the second PPG (BS2) in step 1001.

At the second location, the processor 420 may acquire the third PPG signal (BS3) and the fourth PPG signal (BS4) through the first sensor 440 and the camera module 450.

The processor 420 may acquire the second PTT (PTT2) for the second location based on the third PPG signal (BS3) and the fourth PPG signal (BS4) in step 1003.

The processor 420 may acquire information (HI) on the height difference between the first location and the second location through the second sensor 460 (for example, an acceleration sensor and/or an angular speed sensor). The processor 420 may determine the height difference (h) between the first location and the second location based on the information (HI) on the height difference in step 1005.

For example, the processor 420 may determine acceleration for the movement from the first location to the second location through the second sensor 460 (the acceleration sensor and/or the angular speed sensor) and acquire the information (HI) on the height difference between the first location and the second location corresponding to the acceleration for the movement. The processor 420 may determine the height difference (h) between the first location and the second location based on the information (HI) on the height difference.

The processor 420 may determine a blood pressure difference between the first location and the second location based on the height difference (h) between the first location and the second location. For example, the processor 420 may determine the blood pressure difference according to the height difference (h) based on “Equation (2)” of FIG. 4B.

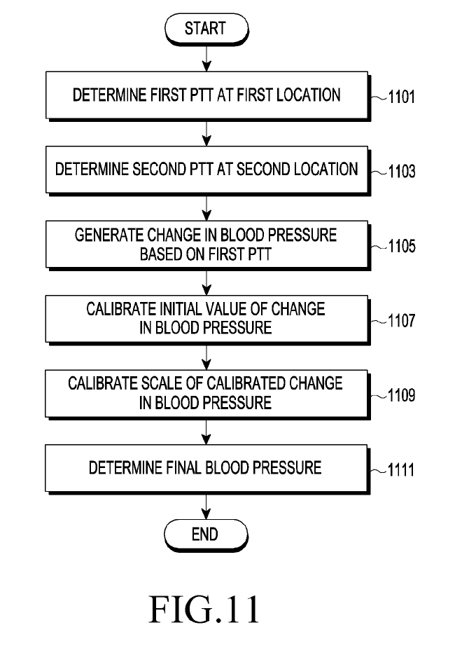

FIG. 11 illustrates operations of a method of determining the blood pressure based on first biometric information and second biometric information according to various embodiments.

Referring to the non-limiting example of FIG. 11, the processor 420 (for example, the processor 420 of FIG. 4A) may determine the first PTT (PTT1) at the first location in step 1101.

The processor 420 may determine the second PTT (PTT2) at the second location according to the user's motion in step 1103.

The processor 420 may generate a change in the blood pressure (or a change in the blood pressure according to the time) based on the first PTT (PTT1) in step 1105. For example, the processor 420 may generate the change in the blood pressure in a graph form (for example, a blood pressure change graph). Here, the blood pressure change graph may mean a blood pressure change according to the time before calibration is performed. Further, the blood pressure change graph may be a value different from the actual blood pressure.

The processor 420 may calibrate an initial value of the blood pressure change (or the blood pressure change graph) in step 1107. For example, the processor 420 may calibrate the initial value of the blood pressure change (or the blood pressure change graph) to the initial blood pressure (IBP).

The processor 420 may calibrate a scale of the calibrated blood pressure change (or the blood pressure change graph) in step 1109. For example, the processor 420 may calibrate a scale of a calibrated first blood pressure change (or a blood pressure change graph) based on the height difference (h) between the first location and the second location and the difference between the first PTT (PTT1) and the second PTT (PTT2).

The processor 420 may determine the blood pressure change (or blood pressure change graph) of which the scale is calibrated as a final blood pressure (BP) in step 1111. The processor 420 may determine a maximum blood pressure and a minimum blood pressure of the blood pressure change (or blood pressure change graph) of which the scale is calibrated.

The processor 420 may display the measured final blood pressure (BP) on the display 470.

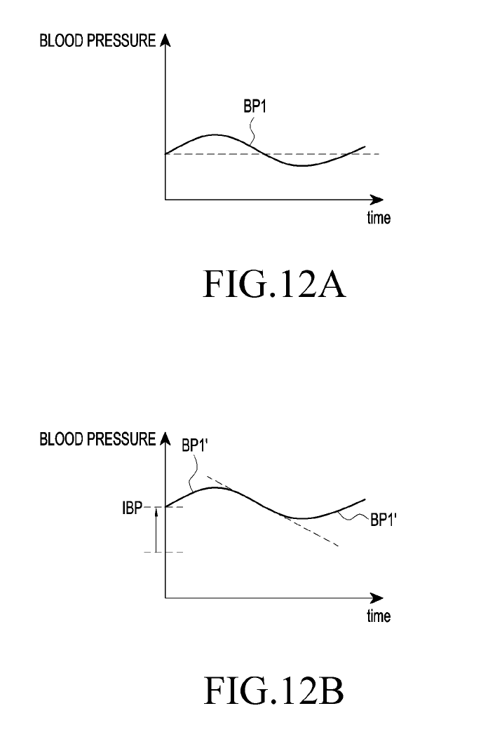

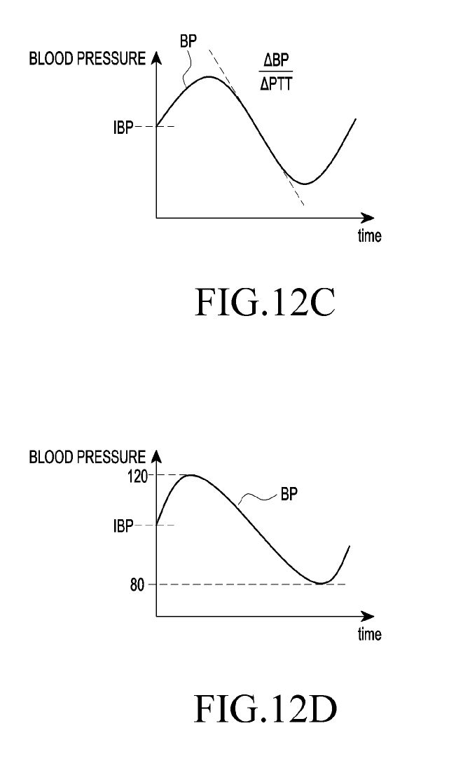

FIGS. 12A to 12D are graphs illustrating aspects of determining the blood pressure based on first biometric information and second biometric information according to various embodiments.

Referring to the non-limiting example of FIG. 12A, the processor 420 may generate a first blood pressure (BP1) in a graph form.

According to an embodiment, the processor 420 (for example, the processor 420 of FIG. 4A) may generate a blood pressure change (or a blood pressure change graph) (BP1) according to the time based on the first PTT (PTT1). For example, the blood pressure change graph (BP1) may mean a blood pressure change graph according to the time before calibration is performed. Further, the blood pressure change graph may include a value different from the actual blood pressure.

The processor 420 may generate the blood pressure change graph (BP1) according to the time based on F(PTT1) of Equation (1) described in FIG. 4B.

Referring to the non-limiting example of FIG. 12B, the processor 420 may calibrate an initial value (or an offset) of the blood pressure change graph (BP1). For example, the processor 420 may calibrate the initial value (for example, a y intercept of the graph) of the blood pressure change graph (BP1) to the initial blood pressure value (IBP).

The processor 420 may generate a blood pressure change graph (BP1′) having an initial value calibrated from the blood pressure change graph (BP1).

Referring to the non-limiting example of FIG. 12C, the processor 420 may calibrate the scale of the calibrated blood pressure change graph (BP1′).

The processor 420 may calibrate the scale of the calibrated blood pressure change graph (BP1′) by controlling the inclination of the calibrated blood pressure change graph (BP1′) between a high point and a low point. For example, the processor 420 may calibrate the scale of the calibrated blood pressure change graph (BP1′) based on the blood pressure difference (ΔBP) acquired through the height difference (h) between the first location and the second location and the difference (ΔPTT) between the first PTT (PTT1) and the second PTT (PTT2).

For example, the processor 420 may acquire ΔBP based on the height difference (h) between the first location and the second location and acquire ΔPTT based on the difference between the first PTT (PTT1) and the second PTT (PTT2).

The processor 420 may calibrate the scale of the calibrated blood pressure change graph (BP1′) based on an inclination obtained by dividing ΔBP by ΔPTT. For example, the processor 420 may calibrate the scale of the calibrated blood pressure change graph (BP1′) by controlling the value obtained by dividing ΔBP by ΔPTT to be an inclination of an intermediate point of the high point and the low point of the calibrated blood pressure change graph (BP1′).

Referring to the non-limiting example of FIG. 12D, the processor 420 may determine the blood pressure change graph of which the scale is calibrated as the final blood pressure (BP). For example, in the graph indicating the final blood pressure (BP), the processor 420 may determine that the high point is a systolic blood pressure and that the low point is a diastolic blood pressure. For example, the processor 420 may determine that the user's blood pressure is 120/80 mmHg.

The processor 420 may display the measured final blood pressure (for example, 120/80 mmHg) on the display 470. Further, the processor 420 may display the graph indicating the measured final blood pressure on the display 470.

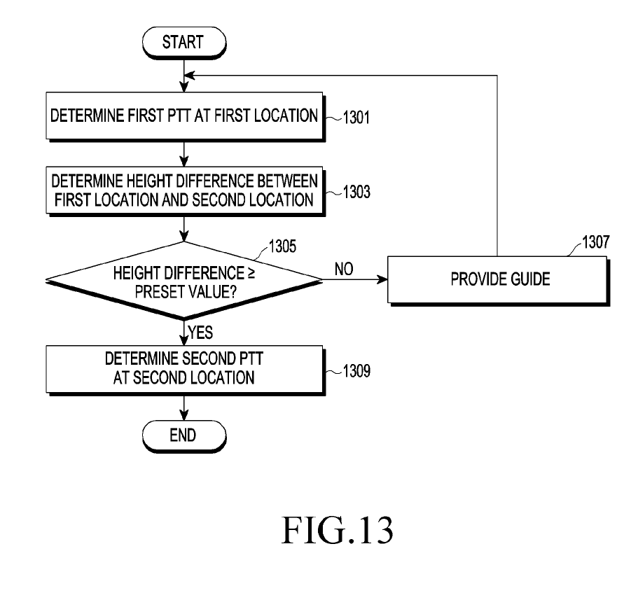

FIG. 13 illustrates operations of a method of acquiring biometric signals at the first location and the second location according to various embodiments.

Referring to the non-limiting example of FIG. 13, the processor 420 (for example, the processor 420 of FIG. 4A) may acquire the first PTT (PTT1) at the first location in step 1301. For example, the processor 420 may acquire the first PTT (PTT1) for the first location based on the first PPG signal (BS1) and the second PPG signal (BS2) acquired through the first sensor 440 and the camera module 450.

When the electronic device 401 (for example, the electronic device 401 of FIG. 4A) moves from the first location to the second location, the processor 420 may determine the height difference between the first location and the second location in step 1303. For example, the processor 420 may determine the height difference between the first location and the second location corresponding to the movement of the electronic device 401 through the second sensor 460.

The processor 420 may compare the determined height difference with a preset value in step 1305. For example, in order to acquire sufficient height difference to determine the blood pressure, the processor 420 may compare the height difference between the first location and the second location with the preset value.

When the height difference between the first location and the second location is smaller than the preset value (No in step 1305), the processor 420 may provide a guide to reset the second location in step 1307. For example, the processor 420 may provide guide information inducing the movement of the electronic device 401 to a second location having a larger height difference from the first location. For example, the processor 420 may display the guide information on the display 470. Further, the processor 420 may provide the guide information through a light, vibration, and/or sound output from the output device 480.

When the height difference between the first location and the second location is larger than or equal to the preset value (Yes in step 1305), the processor 420 may acquire the second PTT (PTT2) at the second location in step 1309. For example, the processor 420 may acquire the second PTT (PTT2) for the second location based on the third PPG signal (BS3) and the fourth PPG signal (BS4) acquired through the first sensor 440 and the camera module 450.

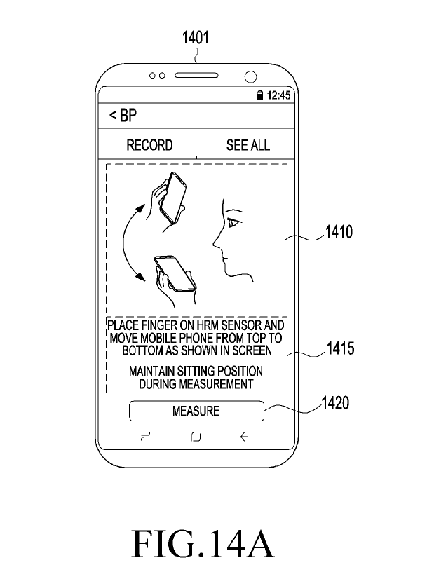

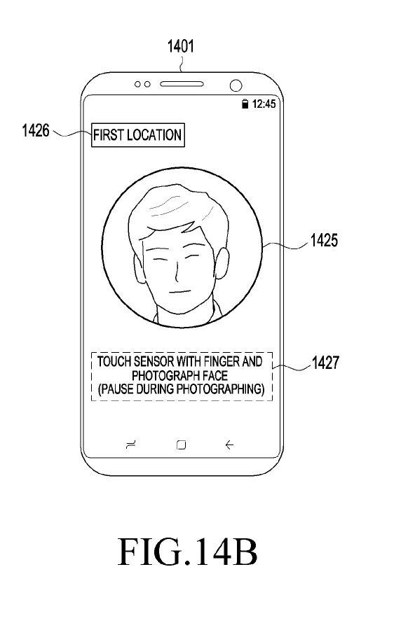

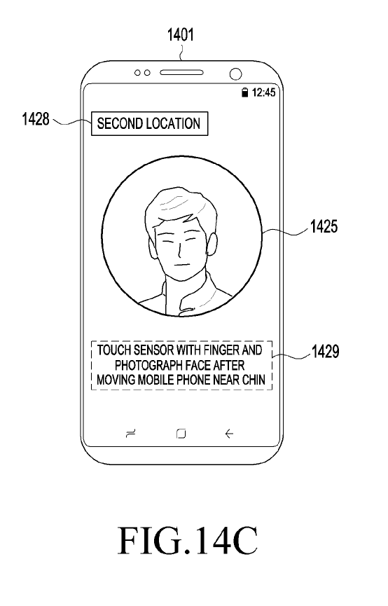

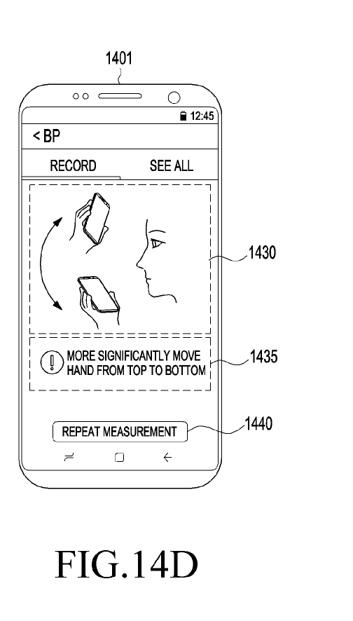

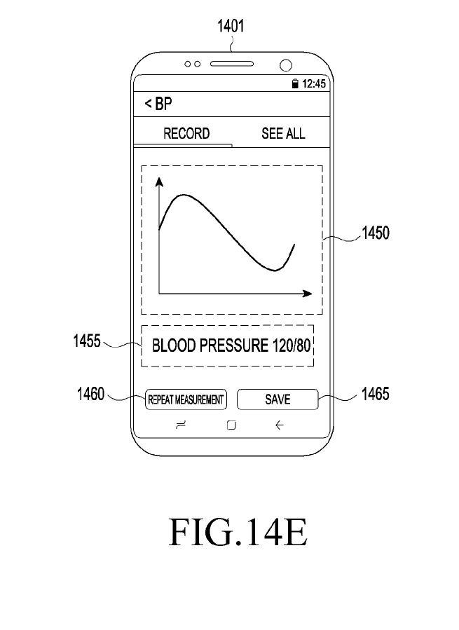

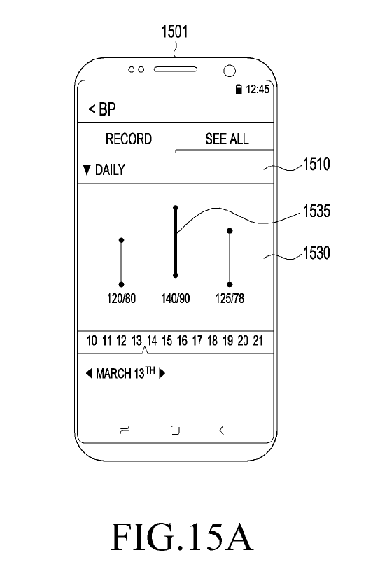

FIGS. 14A to 14E illustrate examples of user interfaces for describing the operation of measuring the blood pressure according to various embodiments.

Referring to the non-limiting example of FIGS. 14A to 14E, the electronic device 1401 may be implemented to be substantially the same or similar to the electronic device 401 of FIG. 4A.

Referring to FIG. 14A, when the user's blood pressure is measured, the electronic device 1401 may provide a measurement method through a user interface. Further, the electronic device 1401 may provide the measurement method through a voice or another multimedia content 1410.

According to an embodiment, the electronic device 1401 may provide the user with the multimedia content 1410 indicating the operation of measuring the blood pressure. For example, the electronic device 1401 may provide the user with the multimedia content 1410 indicating the operation of moving the electronic device to measure the blood pressure.

The electronic device 1401 may provide the user with guide information 1415 that induces the user to “touch the first sensor 440 with a part of the user's body (for example, with the fingers)”. For example, when the user's finger touches the first sensor 440, the electronic device 1401 may provide a first notification through a sound, light, and/or vibration output from the output device 480. Further, when the user's finger does not touch the first sensor 440, the electronic device 1401 may provide a second notification through a sound, light, and/or vibration different from the first notification.

The electronic device 1401 may provide the user with the guide information 1415 for inducing movement of the electronic device 1401 “from top to bottom” or “from bottom to top”. Further, the electronic device 1401 may provide the guide information 1415 for inducing the user to maintain a sitting position during the measurement.

The electronic device 1401 may display an object 1420 corresponding to “measure”. For example, in response to an input for the object 1420 corresponding to “measure”, the electronic device 1401 may perform the operation of measuring the user's blood pressure.

Referring to FIG. 14B, the electronic device 1401 may provide guide information 1427 for inducing the user to “hold still in order to photograph the user's face while maintaining the state in which the user's finger touches the first sensor 440”.

The electronic device 1401 may provide an object 1426 indicating the photographing state at the first location.

Further, when the user's face is recognized in a camera recognition region 1425 according to the guide information and an ROI is determined, the electronic device 1401 may provide a notification through a sound, light, and/or vibration.

Referring to FIG. 14C, the electronic device 1401 may provide guide information 1429 for inducing the user to “move the electronic device near the chin while maintaining the state where the user's finger touches the first sensor 440”.

The electronic device 1401 may provide an object 1428 indicating a photographing state at the second location.

Further, when the user's face is recognized in a camera recognition region 1425 according to the guide information and an ROI is determined, the electronic device 1401 may provide a notification through a sound, light, and/or vibration.

Referring to FIG. 14D, the electronic device 1401 may provide guide information 1435 for inducing the user to “move the electronic device 401 a greater distance while maintaining the state in which the user's finger touches the first sensor 440”. Further, the electronic device 1401 may provide the user with multimedia content 1430 indicating the operation of moving the electronic device to measure the blood pressure.

In addition, the electronic device 1401 may provide guide information for inducing “repeat measurement”.

For example, when a sufficient height difference is not detected through the second sensor 460, the electronic device 1401 may provide a second notification with a sound, light, and/or vibration through the output device 480. At this time, the second notification may be different from the first notification, which is generated when a sufficient height difference is detected through the second sensor 460.

The electronic device 1401 may display an object 1440 corresponding to “repeated measurement”. For example, in response to an input for the object 1440 corresponding to “repeated measurement”, the electronic device 1401 may perform the operation of measuring the user's blood pressure again.

Referring to FIG. 14E, when the measurement of the blood pressure is completed, the electronic device 1401 may display the measured blood pressure 1455 on the display 470 (for example, the display 470 of FIG. 4A).

According to an embodiment, the electronic device 1401 may provide the measured blood pressure in a graph form 1450. Further, the electronic device 1401 may provide the blood pressure 1455 including a systolic blood pressure (for example, 120) and a diastolic blood pressure (for example, 80).

The electronic device 1401 may display an object 1460 corresponding to “repeated measurement” and an object 1465 corresponding to “save”. For example, in response to an input for the object 1460 corresponding to “repeat measurement”, the electronic device 1401 may measure the user's blood pressure again. Further, in response to an input for the object 1465 corresponding to “save”, the electronic device 1401 may store the measured blood pressure or transmit the measured blood pressure to another electronic device.

Meanwhile, the electronic device 1401 may provide guide information illustrated in FIGS. 14A to 14D whenever the blood pressure is measured, or may provide guide information only once at first.

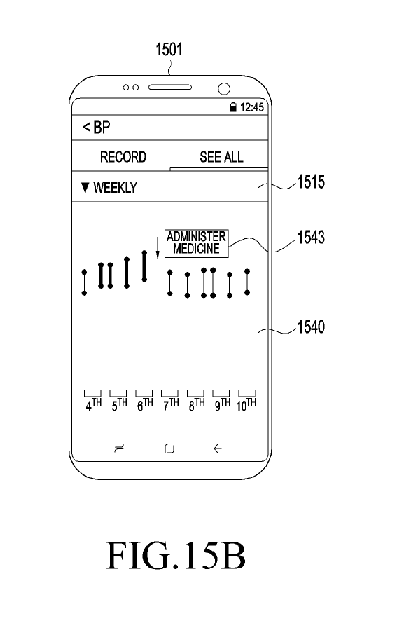

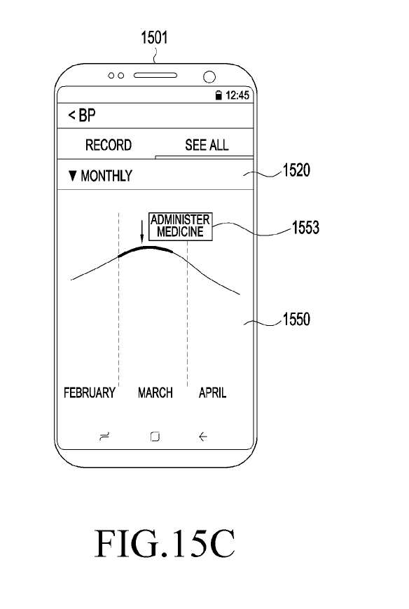

FIGS. 15A to 15C illustrate examples of user interfaces for describing the operation of storing the blood pressure according to various embodiments.

Referring to the non-limiting example of FIGS. 15A to 15C, the electronic device 1501 may be implemented to be substantially the same or similar to the electronic device 401 of FIG. 4A. The electronic device 1501 may individually store the measured blood pressures for each day, week, and month.

Referring to FIG. 15A, the electronic device 1501 may individually store and provide the measured blood pressures for each day.

According to an embodiment, the electronic device 1501 may individually display the measured blood pressures for each day on the display 470 (for example, the display 470 of FIG. 4A).

The electronic device 1501 may display a status bar 1510 indicating that the blood pressures are displayed for each day.

The electronic device 1501 may display information 1530 on a plurality of blood pressures measured on the corresponding day on the display 470.

Further, the electronic device 1501 may determine whether the measured blood pressure is high blood pressure or low blood pressure. For example, when the measured blood pressure is high blood pressure (or low blood pressure), the electronic device 1501 may display the blood pressure in a manner that distinguishes the same from other blood pressures. For example, when the measured blood pressure 1535 is high blood pressure, the electronic device 1501 may display the measured blood pressure 1535 so as to distinguish it from other blood pressures using a color, form, shape, and/or a separate object.

When the systolic blood pressure is higher than or equal to 140 and the diastolic blood pressure is higher than or equal to 90, the electronic device 1501 may determine that the blood pressure is high blood pressure. Further, when the systolic blood pressure is lower than 90 and the diastolic blood pressure is lower than 60, the electronic device 1501 may determine that the blood pressure is low blood pressure.

According to an embodiment, when the measured blood pressure is high blood pressure or low blood pressure, the electronic device 1501 may provide a separate notification window or notification object. For example, the electronic device 1501 may display the separate notification window or notification object on the display 470. Further, the electronic device 1501 may provide a notification through vibration, sound, and/or light through the output device 480.

Referring to the non-limiting example of FIG. 15B, the electronic device 1501 may individually display the measured blood pressures for each week on the display 470.

The electronic device 1501 may display a status bar 1515 indicating that the blood pressures are displayed for each week.

The electronic device 1501 may display information 1540 on a plurality of blood pressures measured on the corresponding week on the display 470.

The electronic device 1501 may display an object 1543 indicating a time point at which the user takes medicine of high blood pressure (or low blood pressure).

Referring to FIG. 15C, the electronic device 1501 may individually display the measured blood pressures for each month on the display 470.

The electronic device 1501 may display a status bar 1520 indicating that the blood pressures are displayed for each month.

The electronic device 1501 may display information 1550 on a plurality of blood pressures measured for each month on the display 470. For example, the electronic device 1501 may display the plurality of blood pressures measured for each month in a graph form.

The electronic device 1501 may display an object 1553 indicating the time point at which the user takes medicine for high blood pressure (or low blood pressure).

According to an embodiment, the electronic device 1501 may set one (for example, an intermediate value) of the previously measured blood pressures as the initial blood pressure (IBP). Further, the electronic device 1501 may set an average value of the previously measured blood pressures as the initial blood pressure (IBP).

An electronic device according to various embodiments may include a first sensor, a camera, and a processor functionally connected to the first sensor and the camera, wherein the processor is configured to acquire a first biometric signal through the first sensor and a second biometric signal through the camera at a first location, acquire a third biometric signal through the first sensor and a fourth biometric signal through the camera at a second location, and determine a blood pressure based on the first biometric signal and the second biometric signal acquired at the first location and the third biometric signal and the fourth biometric signal acquired at the second location.

The processor may be configured to acquire first biometric information based on a difference between the first biometric signal and the second biometric signal, acquire second biometric information based on a difference between the third biometric signal and the fourth biometric signal, and determine the blood pressure based on the first biometric information and the second biometric information.

The first biometric information may include a first pulse transit time based on the difference between the first biometric signal and the second biometric signal and a second pulse transit time based on the difference between the third biometric signal and the fourth biometric signal, and the processor may be configured to determine the blood pressure based on a difference between the first pulse transit time and the second pulse transit time.

The electronic device may further include a second sensor, and the processor may be configured to determine a height difference between the first location and the second location through the second sensor and determine the blood pressure based on the determined height difference.

The processor may be configured to compare the height difference with a preset value and determine the blood pressure according to a result of the comparison.

The processor may be configured to determine the blood pressure based on the height difference when the height difference is larger than or equal to the preset value, and acquire the second biometric information again when the height difference is smaller than the preset value.

The processor may be configured to acquire the second biometric signal from images obtained through the camera.

The processor may be configured to determine a region of interest included in the images and acquire the second biometric signal based on a change in the determined region of interest.

The processor may be configured to acquire the first biometric information by comparing peak points of the first biometric signal and peak points of the second biometric signal.

The processor may be configured to determine that an intermediate value of comparison values obtained by comparing the peak points of the first biometric signal and the peak points of the second biometric signal is the first pulse transit time.

The electronic device may further include a memory, and the processor may be configured to determine the blood pressure based on an initial blood pressure stored in the memory.

The first location and the second location may have different heights.

Each of the first biometric signal, the second biometric signal, the third biometric signal, and the fourth biometric signal may include a PPG signal.

A method of operating an electronic device may include an operation of acquiring a first biometric signal through the first sensor included in the electronic device and a second biometric signal through the camera included in the electronic device at a first location, an operation of acquiring a third biometric signal through the first sensor and a fourth biometric signal through the camera at a second location, and an operation of determining a blood pressure based on the first biometric signal and the second biometric signal acquired at the first location and the third biometric signal and the fourth biometric signal acquired at the second location.

The operation of acquiring the first biometric signal and the second biometric signal may include an operation of acquiring first biometric information based on a difference between the first biometric signal and the second biometric signal, the operation of acquiring the third biometric signal and the fourth biometric signal may include an operation of acquiring second biometric information based on a difference between the third biometric signal and the fourth biometric signal, and the operation of determining the blood pressure may include an operation of determining the blood pressure based on the first biometric information and the second biometric information.

The first biometric information may include a first pulse transit time based on the difference between the first biometric signal and the second biometric signal and a second pulse transit time based on the difference between the third biometric signal and the fourth biometric signal, and the processor may be configured to determine the blood pressure based on a difference between the first pulse transit time and the second pulse transit time.

The operation of determining the blood pressure may include an operation of determining a height difference between the first location and the second location through the second sensor included in the electronic device and determining the blood pressure based on the determined height difference.

The operation of determining the blood pressure may include an operation of determining the blood pressure based on an initial blood pressure stored in a memory of the electronic device.

The method may further include an operation of determining whether the blood pressure is a high blood pressure or a low blood pressure and displaying a result of the determination on a display of the electronic device.

A computer-readable recording medium according to various embodiments may perform an operation of acquiring a first biometric signal through the first sensor and a second biometric signal through the camera at a first location, an operation of acquiring a third biometric signal through the first sensor and a fourth biometric signal through the camera at a second location, and an operation of determining a blood pressure based on the first biometric signal and the second biometric signal acquired at the first location and the third biometric signal and the fourth biometric signal acquired at the second location.

Each of the components of the electronic device according to the present disclosure may be implemented by one or more components and the name of the corresponding component may vary depending on a type of the electronic device. In various embodiments, the inspection apparatus may include at least one of the above-described elements. Some of the above-described elements may be omitted from the electronic device, or the inspection apparatus may further include additional elements. Further, some of the components of the electronic device according to the various embodiments may be combined to form a single entity, and thus, may equivalently execute functions of the corresponding elements prior to the combination.

Various embodiments disclosed herein are provided merely to easily describe technical details of the present disclosure and to help the understanding of the present disclosure, and are not intended to limit the scope of the present disclosure. Therefore, it should be construed that all modifications and changes or modified and changed forms based on the technical idea of the present disclosure fall within the scope of the present disclosure.

Although the present disclosure has been described with various embodiments, various changes and modifications may be suggested to one skilled in the art. It is intended that the present disclosure encompass such changes and modifications as fall within the scope of the appended claims.

What is claimed is:

1. An electronic device comprising:
a first sensor;
a camera; and
a processor functionally connected to the first sensor and the camera,
wherein the processor is configured to:
acquire a first biometric signal through the first sensor and a second biometric signal through the camera at a first location,
acquire a third biometric signal through the first sensor and a fourth biometric signal through the camera at a second location, and
determine a blood pressure based on the first biometric signal and the second biometric signal acquired at the first location and the third biometric signal and the fourth biometric signal acquired at the second location.

2. The electronic device of claim 1, wherein the processor is configured to:
acquire first biometric information based on a difference between the first biometric signal and the second biometric signal,
acquire second biometric information based on a difference between the third biometric signal and the fourth biometric signal, and
determine the blood pressure based on the first biometric information and the second biometric information.

3. The electronic device of claim 2, wherein the first biometric information includes a first pulse transit time based on the difference between the first biometric signal and the second biometric signal and a second pulse transit time based on the difference between the third biometric signal and the fourth biometric signal, and the processor is configured to determine the blood pressure based on a difference between the first pulse transit time and the second pulse transit time.

4. The electronic device of claim 2, further comprising a second sensor, wherein the processor is configured to:
determine a height difference between the first location and the second location through the second sensor and
determine the blood pressure based on the determined height difference.

5. The electronic device of claim 4, wherein the processor is configured to:
compare the height difference with a preset value and determine the blood pressure according to a result of the comparison of the height difference with the preset value.

6. The electronic device of claim 5, wherein the processor is configured to:
determine the blood pressure based on the height difference when the height difference is larger than or equal to the preset value, and
acquire the second biometric information again when the height difference is smaller than the preset value.

7. The electronic device of claim 1, wherein the processor is configured to acquire the second biometric signal from images obtained through the camera.

8. The electronic device of claim 7, wherein the processor is configured to:
determine a region of interest included in the images and
acquire the second biometric signal based on a change in the determined region of interest.

9. The electronic device of claim 2, wherein the processor is configured to acquire the first biometric information by comparing peak points of the first biometric signal and peak points of the second biometric signal.

10. The electronic device of claim 9, wherein the processor is configured to determine that an average value or an intermediate value of comparison values obtained by comparing the peak points of the first biometric signal and the peak points of the second biometric signal is a first pulse transit time.

11. The electronic device of claim 1, further comprising a memory, wherein the processor is configured to determine the blood pressure based on an initial blood pressure stored in the memory.

12. The electronic device of claim 1, wherein the first location and the second location have different heights.

13. The electronic device of claim 1, wherein each of the first biometric signal, the second biometric signal, the third biometric signal, and the fourth biometric signal includes a photoplethysmogram (PPG) signal.

14. A method of operating an electronic device, the method comprising:
acquiring a first biometric signal through a first sensor included in the electronic device and a second biometric signal through a camera included in the electronic device at a first location;
acquiring a third biometric signal through the first sensor and a fourth biometric signal through the camera at a second location; and
determining a blood pressure based on the first biometric signal and the second biometric signal acquired at the first location and the third biometric signal and the fourth biometric signal acquired at the second location.

15. The method of claim 14, wherein the acquiring of the first biometric signal and the second biometric signal comprises acquiring first biometric information based on a difference between the first biometric signal and the second biometric signal, the acquiring of the third biometric signal and the fourth biometric signal comprises acquiring second biometric information based on a difference between the third biometric signal and the fourth biometric signal, and the determining of the blood pressure comprises determining the blood pressure based on the first biometric information and the second biometric information.

16. The method of claim 15, wherein the first biometric information includes a first pulse transit time based on the difference between the first biometric signal and the second biometric signal and a second pulse transit time based on the difference between the third biometric signal and the fourth biometric signal, and the electronic device is configured to determine the blood pressure based on a difference between the first pulse transit time and the second pulse transit time.

17. The method of claim 14, wherein the determining the blood pressure comprises determining a height difference between the first location and the second location through a second sensor included in the electronic device and determining the blood pressure based on the determined height difference.

18. The method of claim 14, wherein the determining the blood pressure comprises determining the blood pressure based on an initial blood pressure stored in a memory of the electronic device.

19. The method of claim 14, further comprising determining whether the blood pressure is a high blood pressure or a low blood pressure and displaying a result of the determination on a display of the electronic device.

20. A computer-readable recording medium comprising program code, which when executed by a processor, causes an electronic device to:
acquire a first biometric signal through a first sensor included in the electronic device and a second biometric signal through a camera included in the electronic device at a first location;
acquire a third biometric signal through the first sensor and a fourth biometric signal through the camera at a second location; and
determine a blood pressure based on the first biometric signal and the second biometric signal acquired at the first location and the third biometric signal and the fourth biometric signal acquired at the second location.
